(19) United States
(12) Patent Application Publication
(10) Pub. No.: US 2025/0041487 A1
(43) Pub. Date: Feb. 6, 2025

(54) Hybrid Tissue Engineering Constructs

(71) Applicant: The Board of Trustees of the Leland Stanford Junior University, Stanford, CA (US)

(72) Inventors: Yunzhi Yang, Stanford, CA (US); Seyedsina Moeinzadeh, Mountain View, CA (US)

(21) Appl. No.: 18/717,323

(22) PCT Filed: Dec. 9, 2022
(86) PCT No.: PCT/US2022/052407

§ 371 Date: Jun. 6, 2024

Related U.S. Application Data
(60) Provisional application No. 63/289,431, filed on Dec. 14, 2021.
(60) Provisional application No. 63/304,216, filed on Jan. 28, 2022.
(60) Provisional application No. 63/289,447, filed on Dec. 14, 2021.
(60) Provisional application No. 63/304,207, filed on Jan. 28, 2022.

(51) Int. Cl. A61L 27/52; A61L 27/34; A61L 27/54; A61L 27/56

(52) U.S. Cl. CPC A61L 27/52; A61L 27/34; A61L 27/54; A61L 27/56; A61L 2400/18; A61L 2420/02; A61L 2420/08; A61L 2430/02

(57) ABSTRACT

Tissue engineering constructs and the method of making the constructs are provided. The constructs distinguish a scaffold with a surface and a treated surface area for increased surface area. A hydrophilic hydrogel network is physically cross-linked via charged polymers and salt-ions onto the treated surface area. Biologies is trapped and thereby hosted within the physically cross-linked hydrogel network. Covalently reactive macromonomers are chemically cross-linked within the physically cross-linked hydrophilic hydrogel network to strengthen the physically cross-linked hydrophilic hydrogel network itself and to the scaffold. The constructs enable delivery of therapeutics including cells and/or biomolecules along with a structural support and a defined geometry for applications in regenerative medicine.

### FIELD OF THE INVENTION

This invention relates to tissue engineering constructs and methods of making thereof.

### BACKGROUND OF THE INVENTION

The regeneration of large bone defects caused by skeletal injuries, diseases, or congenital disorders remains a significant clinical problem. Over 0.5 million and 2 million bone grafting procedures are done in the US and worldwide, respectively every year. Autologous bone is the gold standard for bone grafting. However, an additional surgery is needed to harvest the autologous bone from the donor site, the amount of harvested bone is limited for reconstruction of large defects and the donor site may become morbid. Allogenic grafts have been widely used as an alternative to autologous grafts for bone regeneration. However, the long-term failure rate of allogenic grafts in treatment of large critical bone defects is 25%˜60% due to various complications. In addition, the use of frozen allografts suffers from a potential risk of disease transmission according to the Center for Disease Control and Prevention (CDC). Commercially available demineralized bone matrix (DBM) contains osteo-inductive factors but DBM alone does not provide the structural and mechanical support for reconstruction of large bone defects. Therefore, synthetic scaffolds as bone graft substitutes have attracted attention in recent years.

An ideal scaffold for bone tissue engineering is biocompatible, bioresorbable, mechanically stable, porous, osteo-conductive and osteo-inductive. Biocompatible, bioresorbable, and FDA-cleared polyesters including polycaprolactone (PCL), polyglycolic acid (PGA), and polylactic acid (PLA) and their copolymers (e.g. PLGA) are the most widely used synthetic polymers in bone tissue engineering. Several methods such as molding, solvent casting/porogen leaching, gas foaming, laser drilling and 3D printing have been applied to make porous polyester-based scaffolds. Among these techniques, 3D printing offers a precise control over the architecture and porosity of the scaffold. A well-controlled porosity of 3D printed scaffolds is particularly important for bone tissue engineering, because the presence of interconnected pores with individual pore size larger than 300 μm is essential for cell migration and bone ingrowth.

The osteo-conductivity and mechanical properties of the polyester-based scaffolds have been augmented by incorporation of calcium phosphate bioceramics. For example, the osteogenic differentiation of mouse preosteoblast cells (MC3T3-E1) on 3D printed PCL/β-tricalcium phosphate (TCP) substrates was significantly higher than on pristine PCL substrates. The inventors, previously, showed that the Young's modulus of 3D printed PCL-TCP scaffolds was tunable in 12 to 188 MPa range by changing the TCP content and scaffold porosity. Further, the clinically available electron beam sterilization did not adversely affect the mechanical and bioactive properties of PCL-TCP scaffolds.

Although 3D printed polymer/ceramic scaffolds are biocompatible, bioresorbable, mechanically stable, porous and osteo-conductive, they lack the osteo-inductive factors to stimulate osteogenic differentiation and accelerate bone healing. Therefore, there is a need to incorporate osteo-inductive proteins into 3D printed scaffolds particularly for treatment of large bone defects. Surface coating has been used to immobilize proteins on the surface of 3D printed scaffolds for tissue engineering applications. However, the loading of proteins on thin coatings is typically limited and the release rate is fast. For instance, the loading of BSA on 3D printed hydroxyapatite-based scaffolds coated with chitosan and sodium hyaluronate by layer-by-layer (LBL) deposition, was lower and the release was faster than uncoated scaffolds.

Hydrogels have been used for protein delivery or cell encapsulation in tissue engineering applications. A 3D polymeric network of hydrogels with a large water content provides a platform for adequate loading and sustained release of proteins. However, inferior stiffness and structural integrity of hydrogels limit their use as stand-alone 3D scaffolds. In addition, incorporation of soft hydrogels into rigid 3D printed scaffolds is challenging due to mechanical property mismatch at the interface. Filling porous 3D printed scaffolds with hydrogel precursor solution followed by photo or thermal induced gelation has been used to incorporate hydrogels into rigid scaffolds. For example, a surface tension-assisted method was used to fill the pores of 3D printed constructs with photo-crosslinkable methacrylated gelatin hydrogel. Multi-material 3D printing has been used to manufacture porous polymer/hydrogel composite scaffolds.

Preservation of the porous structure of the scaffold after incorporation of hydrogel is essential for cell migration, tissue integration, and vascularization for diffusion of nutrients and oxygen in tissue engineering applications. An integrated tissue-organ printer was used to sequentially print a gelatin/fibrinogen-based hydrogel along with PCL structural support. Stanford University developed a 3D hybrid bioprinting technology (Hybprinter) and used it for printing composite scaffolds from PCL and polyethylene glycol diacrylate (PEGDA) hydrogel. Despite its technological significance, multi-material printing requires a long fabrication time due to multiple iterations between the materials during printing, and a specialized expensive 3D printer. Furthermore, concurrent printing of polymer/ceramic and hydrogels hinders the scaffold surface treatment and improving the integration between soft and rigid materials at the interface.

### SUMMARY OF THE INVENTION

This invention in one example provides a porous or a non-porous biologics-loaded multimaterial construct, hereafter referred to as Hybrid Tissue Engineering Construct (HyTEC) for applications in regenerative medicine and treatment of diseases.

Constructs and devices made of polymers, ceramics, metals, or composites in porous or non-porous forms have been widely used as implants in regenerative medicine. A number of techniques, including 3D printing and casting, have been used to manufacture porous implants. Also, coating techniques including layer-by-layer coating or adhesive coating have been used to load biomolecules on the surface of porous implants. However, these coating techniques only allow loading of a limited amount of biomolecules. Loading a large or tunable dose of biomolecules on implants is particularly important since the effective dose of biomolecules is often high in-vivo and could be different for various indications.

Biologics (biomolecules, drugs and/or cells) could be loaded on implants via filling the porous structure of the implant with a biologics-loaded hydrogel. However, filling the porous space of implants with a hydrogel closes the pores and inhibits or mitigates cell recruitment and migration, vascular invasion, tissue regeneration, and integration with surrounding tissues.

To address at least this concern, the inventors of this invention have developed a strategy to engineer a HyTEC that enables incorporation of biologics through a uniform thick hydrogel layer onto porous scaffolds while retaining interconnected open pores, or onto non-porous implants (FIG. 1). For the proof of concept, the inventors loaded model proteins and cells on 3D printed biodegradable polycaprolactone and β-tricalcium phosphate (PCL-TCP) as a model polymer-ceramic porous scaffold, a PCL-TCP rod as a model polymer-ceramic non-porous implant, and stainless-steel needles as a model metal.

The surface of porous or non-porous scaffolds is treated in three consecutive steps to (FIGS. 2A-B):
(1) increase hydrophilicity/reactivity/roughness,
(2) improve hydrogel adhesion, and
(3) stimulate surface-initiated crosslinking.

A layer of hydrogel is loaded on the surface of scaffolds through a surface-initiated physical crosslinking followed by covalent crosslinking.

### Increase Hydrophilicity/Reactivity/Roughness

Sodium hydroxide (NaOH) treatment and freezing/thawing were used to increase the surface hydrophilicity/reactivity in PCL-TCP scaffolds. Other treatment methods including plasma or acid treatment could also be used to increase the surface hydrophilicity/reactivity/roughness.

### Improve Hydrogel Adhesion

For improving the hydrogel adhesion, the surface is coated with a molecule that has a covalently linkable functional group. For instance, reactive Aminopropyl methacrylamide (APMA), and Gelatin methacrylate (GelMA) have been conjugated to the surface of PCL-TCP scaffolds using carbodiimide chemistry (FIGS. 2A-B). Amine reactive (N-hydroxy succinimide) ester diazrine (NHS-diazirine, succinimidyl 4,4′-azipentanoate) was conjugated to the surface of stainless steel.

### Stimulate Surface-Initiated Crosslinking

To stimulate surface-initiated physical crosslinking, calcium chloride (CaCl₂) or calcium sulfate (CaSO₄) was deposited on the surface of the implants. Other salts of divalent cations (e.g. Ca²⁺, Mg²⁺, Sr²⁺) or multivalent cations (e.g. Ti⁴⁺ or Al³⁺) could also be used for surface initiated physical crosslinking.

After these three steps of surface treatment, the scaffolds are dipped into a hydrogel precursor solution containing alginate, covalently reactive macromonomers, an initiator, and biologics (biomolecules, drugs, and/or cells). Polyethylene glycol dimethacrylate (PEGDMA) and GelMA were used as covalently reactive macromonomers (FIGS. 2A-B). Other macromonomers and crosslinkers with double bonds or other covalently reactive functional groups (e.g. NHS group for amine reaction or SH group for Michael addition) could be used in the hydrogel precursor solution.

When the surface treated scaffolds are dipped into the hydrogel precursor solution, calcium ions diffuse from the surface to the solution, crosslink alginate at the proximity of the surface, and make a hydrogel layer on the surface. The macromonomers within the physically crosslinked hydrogel are then covalently crosslinked in the next step to form a stiff interpenetrating network. A chemical initiator (APS/TEMED) and a photoinitiator (Lithium phenyl-2,4,6-trimethylbenzoylphosphinate) have been used for making porous and non-porous HyTECs, respectively (FIGS. 2A-B). Other chemical initiators, visible light initiators, UV initiators, or thermal initiators could be used for initiating the covalent crosslinking reaction.

### Tuning

The hydrogel loading and hydrogel thickness are tuned by changing the process parameters. For instance, the thickness of hydrogel layer on porous PCL-TCP scaffolds was tuned by changing the NaOH surface treatment time and the CaCl2) concentration in the solution that was used for calcium deposition. The coating thickness within a construct could be tuned/controlled from zero to high value spatially by dipping different parts into different solutions.

### Results

The 3D printed porous PCL-TCP scaffolds with different porosities remained porous after hydrogel loading (FIG. 3A). When BMP2 (as a model protein) was loaded on HyTEC, it was released over 28 days ex-vivo. A wide range of biomolecule doses could be loaded on HyTEC due to the presence of a thick hydrogel layer. The biomolecule-laden HyTEC could be freeze-dried, stored, and terminally sterilized using electron beam (E-beam) method with split dose (FIGS. 4A-C). Live cells could be encapsulated in the hydrogel layer of HyTEC (FIGS. 5A-D). Further, hydrogel was loaded on stainless steel needles as a proof of concept for making metal-based HyTECs (FIG. 6).

Depending on the application, the HyTEC can be cellular or cell free, with or without therapeutic biomolecules. It can be fresh, freeze or freeze-dried. The scaffold can also be porous or non-porous and be made from polymers (e.g. polyesters), metals, ceramics, or composites. The hydrogel macromonomer, gelation initiator, salt for calcium deposition, and the material used for surface treatment can be changed depending on the scaffold surface chemistry and application.

### Applications

Embodiments of the invention could be applied or used in the following ways without any limitation to be scope of the invention. HyTEC used for delivery of therapeutics including cells and/or biomolecules along with a structural support and a defined geometry for applications in regenerative medicine. Examples are as follows:
delivery of osteo-inductive proteins and osteogenic cells along with osteo-conductive 3D printed constructs for treatment of bone defects.
delivery of proteins and/or cells along with 3D printed constructs for treatment of soft tissue defects.
delivery of antibiotics along with 3D printed scaffolds.
delivery of painkillers along with 3D printed scaffolds.
delivery of proteins and drugs along with metallic implants.
delivery of vasculo-inductive proteins or cells to induce vascularization in regenerative medicine.
local delivery of therapeutics in cancerous tissues.
local delivery of β cells for insulin secretion in diabetic patients.

### Advantages

Embodiments of the invention are advantageous over existing approaches and constructs. A large dose, a broader spectrum of dose, or a variety of therapeutics or biologics can be loaded on porous (or non-porous) constructs using HyTEC technology as opposed to methods that are based on thin coatings (e.g. layer-by-layer coating or coating the construct with an absorbent). In addition, cells can be encapsulated in HyTEC as opposed to constructs with thin coating.

The advantage of HyTEC technology over multimaterial printing is as follows. Despite its technological significance, multimaterial printing requires a long fabrication time due to multiple iterations between the materials during printing, a specialized expensive 3D printer, and limited selections of processing parameters due to the nature of various printing mechanisms. Furthermore, concurrent printing of polymer/ceramic and hydrogels hinders the scaffold surface treatment and improving the integration between soft and rigid materials at the interface.

In one embodiment, to slow down the release of therapeutics, the bioactive implants (e.g. HyTEC constructs) could be coated with a resorbable polyester (e.g. PCL, PLA, or PLGA) or other resorbable polymers (e.g. polyurethanes). HyTEC stands for hybrid tissue engineered construct, which is a bioactive implant. A schematic representation of the method that is used to coat the HyTEC constructs is shown in FIG. 7. For instance, protein-laden HyTEC is freezed at −80° C. overnight followed by 10 minutes freezing at −20° C., and dipped in a solution of PCL in acetone or chloroform (2%-20%) to deposit a layer of PCL on HyTEC and make modified HyTEC (mHyTEC). Then the PCL-coated HyTEC is air-dried at 0-4° C. The concentration of PCL solution and the number of deposited PCL layers could be changed to tune the physical characteristics of mHyTEC constructs and release kinetics of proteins. Representative images of mHyTEC with 1 layer of PCL coating (mHyTEC (1L)) and 3 layers of PCL coating (mHyTEC (3L)) are shown in FIG. 8. While 92% of the encapsulated BSA was released from BSA-laden HyTEC without coating after 14 days, the rate of BSA release was reduced to 92% after 70 days or 80% in 91 days, with addition of 1 layer or 3 layers of protective PCL coating using a PCL/acetone (10% wt/v) solution (FIG. 9). Also, the amount of released bone morphogenic protein 2 (BMP2) protein from BMP2-laden HyTEC constructs after 28 days in PBS decreased was 84% to 62% or 24% with deposition of 1 layer or 3 layers of PCL coating (FIG. 10).

In another example, the present invention provides a method of forming a tissue engineering construct. A scaffold with a surface and a surface area is provided. The surface of the scaffold is treating to increase the surface area of the scaffold. Optionally the surface area of the scaffold is prepared to facilitate a chemical cross-linking to the surface area by coating the surface area with covalently linkable molecules. The surface area of the scaffold is prepared to facilitate surface-initiated physical cross-linking by depositing a salt onto the surface area or the optionally coated surface area. A hydrogel precursor solution is provided/prepared containing charged polymers, covalently reactive macromonomers, an initiator and biologics. A physically cross-linked hydrophilic hydrogel network is formed onto the surface of the scaffold by immersing the prepared scaffold into a hydrogel precursor solution. The forming is controlled by a release of salt-ions from the surface area and physically cross-linking the charged polymers with the released salt-ions. During the formation the biologics becomes trapped and thereby hosted within the physically cross-linked hydrogel network. The scaffold with the physically cross-linked hydrophilic hydrogel network is removed from the hydrogel precursor solution. The covalently reactive macromonomers are chemically cross-linked within the physically cross-linked hydrophilic hydrogel network to strengthen the physically cross-linked hydrophilic hydrogel network itself and to the scaffold. Optionally (following the optional preparation of the surface area infra) the coated covalently linkable molecules are chemical cross-linked with the covalently reactive macromonomers to increase adhesion of the chemically and physically cross-linked hydrophilic hydrogel network to the scaffold.

In a further step, freeze or freeze-drying of the tissue engineering construct can be performed if needed.

In a further step, the surface area of the scaffold can be treated to increase the hydrophilicity and/or roughness of the surface of the scaffold.

The scaffold can be an interconnected porous structure and as such the methods steps can then be controlled for the hydrophilic hydrogel network to be physically and chemically crosslinked and chemically bound to the interconnected porous structure of the scaffold. The pores of the interconnected porous structure can then be preserved by these controlled method steps to allow the pores to also be house the biologics.

In still a further step, the tissue engineering construct can be coated with one or more coating layers.

In still a further step, the scaffold has an interconnected porous structure and where the coating controls pore size of the interconnected porous structure.

In another embodiment, a tissue engineering construct is provided. The construct distinguished a scaffold with a surface and a treated surface area for increased surface area. A hydrophilic hydrogel network is physically cross-linked via charged polymers and salt-ions onto the treated surface area. Biologics is trapped and thereby hosted within the physically cross-linked hydrogel network. Covalently reactive macromonomers are chemically cross-linked within the physically cross-linked hydrophilic hydrogel network to strengthen the physically cross-linked hydrophilic hydrogel network itself and to the scaffold.

In a variation of the construct, the surface area can be coated with covalently linkable molecules which are chemical cross-linked with the covalently reactive macromonomers to increase adhesion of the chemically and physically cross-linked hydrophilic hydrogel network to the scaffold. The construct can have one or more coatings. The scaffold can be an interconnected porous scaffold where the biologics are then also hosted with pores of the interconnected porous scaffold.

Fused deposition modeling is a powerful method for printing 3 dimensional (3D) bioresorbable scaffolds and medical devices with well-controlled porosity, internal microstructure, and overall geometry for biomedical applications. However, proteins and live cells are not able to withstand the hot extrusion. In a further characterization of the invention a hybrid tissue engineering construct (HyTEC) is engineered that enables incorporation of biologics (e.g. proteins and cells) through a uniform thick hydrogel layer onto 3D printed scaffolds while retaining interconnected open pores, or onto non-porous implants. A 3D printed biodegradable polycaprolactone-β-tricalcium phosphate (PCL-TCP) was used as a model porous scaffold, a PCL-TCP rod as a model non-porous implant, and bone morphogenetic protein-2 (BMP-2) as a model protein for bone tissue engineering application. The surface of PCL-TCP constructs was treated in three consecutive steps to increase hydrophilicity, improve hydrogel adhesion and stimulate surface-initiated crosslinking. A layer of hydrogel was loaded on the surface of scaffolds through a surface-initiated physical crosslinking followed by covalent crosslinking. The results showed that surface treatment did not adversely affect the mechanical and surface properties of the scaffolds but improved the adhesion of hydrogel to the surface. The average thickness of the loaded hydrogel layer was controlled in the range of 100-600 μm by adjusting surface treatment parameters. In addition, 3D printed scaffolds with 50-80% porosity remained porous after hydrogel loading with pore sizes ranged from 140 to 1100 μm. Cell viability and proliferation tests using two cell types (hMSCs and C3H10) showed that hydrogel loading did not adversely influence the biocompatibility of scaffolds. BMP-2-laden hydrogel loaded scaffolds released BMP-2 in a sustained manner over 35 days. Freeze-drying and E-beam sterilization of hydrogel loaded PCL-TCP scaffolds did not adversely affect the mechanical properties of scaffolds but negatively impacted the amount of released active BMP-2. The amount of released active BMP-2 from sterilized freeze-dried HyTEC constructs was improved by 2 folds by using a split dose E-beam strategy. A thick hydrogel layer enabled loading encapsulated live cells on HyTEC constructs with over 92% cell viability after 7 days. In summary, the HyTEC strategy introduced in this study holds great promises in porous (or non-porous) polyester-based 3D printed tissue engineering scaffolds with improved payload capacity of biological substances while maintaining interconnected open pores for improved tissue integration and engraftment.

### BRIEF DESCRIPTION OF THE DRAWINGS

If needed, for further interpretation of the gray-scale in the drawings the reader is referred to the priority document(s) for each of the respective figures.

FIG. 1 shows according to an exemplary embodiment of the invention a schematic representation of HyTEC.

FIGS. 2A-B show according to an exemplary embodiment of the invention in FIG. 2A a schematic representation of the process used to make porous HyTEC, and in FIG. 2B a schematic representation of the process used to make non-porous HyTEC. FIG. 2A, the schematic diagram for manufacturing 3D printed porous PCL-TCP/hydrogel HyTECs constructs, after 3D printing, the PCL-TCP scaffolds were treated with NaOH to increase the surface hydrophilicity caused by the scission of ester bonds to carboxyl and hydroxyl groups. Reactive double bonds were then incorporated onto the surface by grafting APMA to carboxyl groups using carbodiimide chemistry. The scaffolds were then dipped into CaCl2) solution and vacuum dried. When the surface treated scaffolds were dipped into alginate/PEGDMA precursor solution, the deposited calcium diffused from the surface to the solution, crosslinked alginate at the proximity of the surface, and made a hydrogel layer on the surface. The PEGDMA macromonomers within the physically crosslinked hydrogel were covalently crosslinked in the next step to form a stiff interpenetrating network. The hydrogel network bound to the scaffold surface through reaction of double bonds of PEGDMA macromonomers and double bonds of APMA grafted to the scaffold surface.

FIGS. 3A-B shows according to an exemplary embodiment of the invention in FIG. 3A a porous HyTECs with different porosities, and in FIG. 3B the effect of HyTEC porosity on the loaded hydrogel thickness.

FIGS. 4A-C show according to an exemplary embodiment of the invention in FIG. 4A a release kinetics of BMP2 from freeze-dried HyTEC, in FIG. 4B the effect of freeze-dried HyTEC storage at 4° C. for 2 months on release kinetics of BMP2, and in FIG. 4C the effect of E-beam sterilization using split dose on release kinetics of BMP2 from freeze-dried HyTEC.

FIGS. 5A-D show according to an exemplary embodiment of the invention in FIG. 5A an image of the human mesenchymal stem cell-laden non-porous HyTEC, in FIG. 5B live (green) and dead (red) cells encapsulated in a non-porous HyTEC, in FIG. 5C human mesenchymal stem cell (hMSC) viability in HyTEC over 7 days, and in FIG. 5D DNA content of cell-laden HyTEC over 14 days.

FIG. 6 shows according to an exemplary embodiment of the invention a model metal-based HyTEC made on a stainless-steel needle.

FIG. 7 shows according to an exemplary embodiment of the invention a schematic representation of the method that is used to coat the bioactive implants (HyTEC constructs).

FIG. 8 shows according to an exemplary embodiment of the invention images of mHyTEC with 1 layer of PCL coating (mHyTEC (1L)) and 3 layers of PCL coating (mHyTEC (3L)) made using a PCL/acetone (10% wt/v) solution.

FIG. 9 shows according to an exemplary embodiment of the invention release kinetics of BSA from HyTEC constructs without PCL protective coating (ctrl), with 1 layer of PCL coating made using a PCL/chloroform (10% wt/v) solution (PCL/chloroform-1L), with 1 layer of PCL coating made using a PCL/acetone (10% wt/v) solution (PCL/acetone-1L), with 3 layers of PCL coating made using a PCL/chloroform (10% wt/v) solution (PCL/chloroform-3L), and with 3 layer of PCL coating made using a PCL/acetone (10% wt/v) solution (PCL/acetone-3L).

FIG. 10 shows according to an exemplary embodiment of the invention release kinetics of rhBMP2 from HyTEC constructs without PCL protective coating (HyTEC), with 1 layer of PCL coating made using a PCL/acetone (10% wt/v) solution (mHyTEC (1L)), and with 3 layers of PCL coating made using a PCL/acetone (10% wt/v) solution (mHyTEC (3L)).

FIGS. 11A-E show according to an exemplary embodiment of the invention in FIG. 11A-B the effect of APMA concentration in the reaction solution on (FIG. 11A) density of grafted APMA onto the scaffold surface and (FIG. 11B) contact angle of scaffold surface. PCL-TCP scaffolds with porosity ranged from 0% to 80% were 3D printed (FIG. 11B), treated with NaOH and APMA (2.5 mg/mL) and used to evaluate the effect of surface treatment on the mechanical properties of scaffolds. (FIGS. 11D-E) Young's modulus (FIG. 11D) and stress at yield (FIG. 11E) of PCL-TCP scaffolds without surface treatment (untreated, B) and with NaOH/APMA treatment (treated/A—2.5, R). Error bars correspond to means±1 SD for n=3.

FIGS. 12A-D show according to an exemplary embodiment of the invention the effect of NaOH treatment time on (FIG. 12A) hydrogel coating, (FIG. 12B) release of calcium ion from the surface of scaffolds, (FIG. 12C) hydrogel thickness and (FIG. 12D) fraction of filled pores in PCL-TCP scaffolds with 80% porosity. For FIGS. 12A, 12C-D the concentration of CaCl2) in the treatment solution and concentration of PEGDMA in the hydrogel precursor solution was 100 mg/mL and 20% (wt/vol), respectively. Error bars correspond to means±1 SD.

FIGS. 13A-D show according to an exemplary embodiment of the invention the effect of concentration of CaCl2) in the treatment solution on (FIG. 13A) hydrogel coating, (FIG. 13B) release of calcium ion from the surface of scaffolds, (FIG. 13C) hydrogel thickness and (FIG. 13D) fraction of filled pores in PCL-TCP scaffolds with 80% porosity. For FIGS. 13A, 13C-D the NaOH treatment time and concentration of PEGDMA in the hydrogel precursor solution was 60 min and 20% (wt/vol), respectively. Error bars correspond to means±1 SD.

FIGS. 14A-F show according to an exemplary embodiment of the invention the effect of scaffold porosity on (FIG. 14A) hydrogel coating onto interconnected porous scaffold, (FIG. 14B) hydrogel thickness, (FIG. 14C) fraction of filled pores, (FIG. 14D) release of calcium ion from the surface of scaffolds (FIG. 14E) release of calcium ion per unit weight of the scaffold and (FIG. 14F) hydrogel loading porous HyTECs. The NaOH treatment time, concentration of CaCl2) in the treatment solution and concentration of PEGDMA in the hydrogel precursor solution was 60 min, 100 mg/mL, and 20% (wt/vol), respectively. Error bars correspond to means±1 SD.

FIGS. 15A-G show according to an exemplary embodiment of the invention the effects of freeze drying on properties of PCL-TCP scaffolds and HyTECs. (FIGS. 15A-B) Effect of freeze drying on Young's modulus (FIG. 15A) and stress at yield (FIG. 15B) of PCL-TCP scaffolds at different porosities. (FIG. 15C) effect of freeze drying and rehydration on the hydrogel coating in 80% porous HyTECs. (FIGS. 15D-E) Effect of NaOH treatment time on (FIG. 15D hydrogel thickness and (FIG. 15E) fraction of filled pores in 80% porous HyTECs before freeze-drying and after freeze-drying and rehydration. (FIGS. 15F-G) effect of concentration of CaCl₂) in the treatment solution on (FIG. 15F) hydrogel thickness and (FIG. 15G) fraction of filled pores in 80% porous HyTECs before freeze-drying and after freeze-drying and rehydration. For FIGS. 15D-E the concentration of CaCl₂) in the treatment solution and concentration of PEGDMA in the hydrogel precursor solution was 100 mg/mL and 20% (wt/vol), respectively. For FIGS. 15F-G the NaOH treatment time and concentration of PEGDMA in the hydrogel precursor solution was 60 min and 20% (wt/vol), respectively. “An asterisk” represents a statistically significant difference between non freeze-dried and freeze-dried/rehydrated samples. The Error bars correspond to means±1 SD. B=before freeze-drying, F=freeze-dried.

FIGS. 16A-H show according to an exemplary embodiment of the invention characterization of mechanical properties and BMP-2 release kinetics under different conditions of HyTECs. (FIGS. 16F-B) The structure of the 3D printed PCL-TCP device which was used to measure the adhesion of hydrogels to scaffolds. The hydrogel was made within the gap between two concentric cylinders. The hydrogel-incorporated device was then placed on the Instron machine and two bridges connecting inner and outer cylinders were cut and the interfacial stiffness was measured via push-out tests (FIG. 16C). (FIGS. 16D-E) effect of APMA surface treatment (FIG. 16D) and PEGDMA polymer concentration in the hydrogel precursor solution (FIG. 16E) on interfacial stiffness of hydrogel. (FIGS. 16F-G); effect of PEGDMA polymer concentration in the hydrogel precursor solution on (FIG. 16F) hydrogel loading in 80% porous PCL-TCP scaffolds and (FIG. 16G) BMP2 release kinetics. (FIG. 16H) release kinetics of BMP2 from fresh (red), freeze dried (green) and freeze dried and E-beam sterilized (blue) BMP2-laden 80% porous HyTEC. In FIGS. 16F-H, the NaOH treatment time and concentration of CaCl₂) in the treatment solution was 60 min and 100 mg/mL, respectively. Error bars correspond to means±1 SD.

FIGS. 17A-H show according to an exemplary embodiment of the invention cell viability, proliferation, and osteogenic differentiation of HyTECs in vitro. (FIG. 17A) effect of hydrogel loading on normalized viability of hMSCs and C3H10s cultured in preconditioned DMEM medium. (FIGS. 17B-C) effect of hydrogel loading on proliferation of (FIG. 17B) MSCs and (FIG. 17C) C3H10 cells cultured in preconditioned DMEM medium. (FIGS. 17D-E) ALP activity of hMSCs (FIG. 17D) and C3H10 cells (FIG. 17E) cultured in DMEM medium (C), DMEM medium preconditioned with HyTEC without BMP2 (scaffold+gel, P), DMEM medium preconditioned with BMP2-laden HyTEC (scaffold+gel/BMP2, R), DMEM medium supplemented with 1.5 μg/mL BMP2 for 3 days (BMP2 in medium (3 d), B), and DMEM medium supplemented with 214 ng/mL BMP2 for 21 days (BMP2 in medium (21 d), G). “An asterisk” represents a statistically significant difference between the test group and all other groups at that time point. The Error bars correspond to means±1 SD.

FIGS. 18A-I show according to an exemplary embodiment of the invention fabrication and characterization of representative non-porous HyTEC. (FIG. 18A) the procedure for coating non-porous PCL-TCP rods with a bioresorbable hydrogel to make HyTEC (FIGS. 18B-C) SEM images of the surface of hydrogel on non-porous HyTEC (FIG. 18D) Average loading of hydrogel without BMP2 (G) and with BMP2 (R) on treated and calcium deposited PCL-TCP rods (FIG. 18E) release kinetics of BMP2 from freeze-dried BMP2-laden hydrogel loaded PCL-TCP rods without E-beam sterilization (G), after sterilization using a single dose E-beam (B) and after sterilization using split doses of E-beam (R). (FIG. 18F) An image of cell-laden non-porous HyTEC, (FIG. 18G) Live(G)/Dead(R) stained cell-laden non-porous HyTEC, (FIG. 18H) viability of hMSCs encapsulated in non-porous HyTEC, (FIG. 18I) DNA content of cell-laden non-porous HyTEC. The Error bars correspond to means±1 SD.

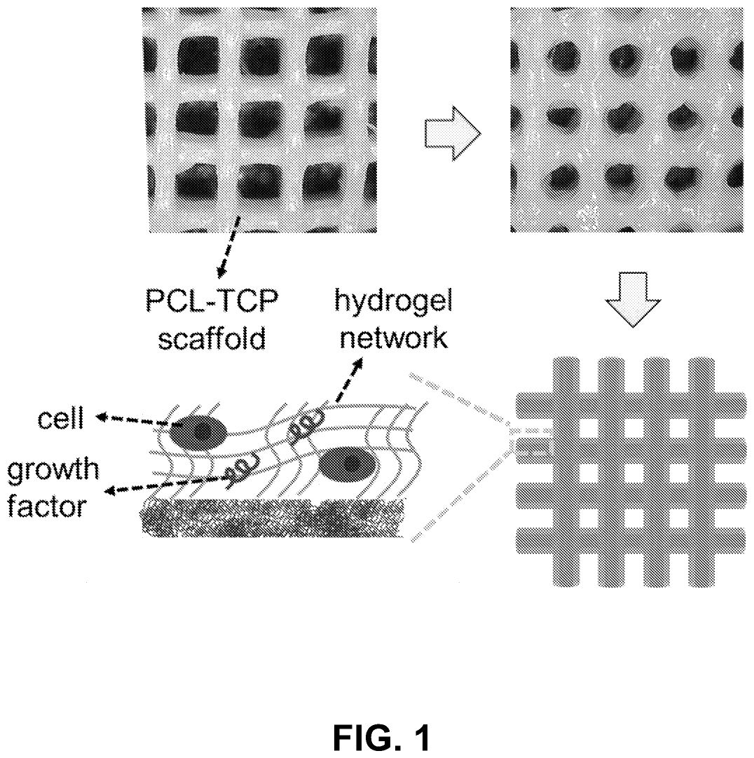

### DETAILED DESCRIPTION

### Definitions

The following detailed description is exemplary embodiments of the method of forming/making the tissue engineering construct and the structural features of the tissue engineering construct. In general, the following definitions of terms can be used as a guidance within the scope of the invention.
A scaffold is defined as a porous or non-porous three-dimensional construct made of polymers, ceramics, metals, or composites.
Treatment of a surface area includes the method of Base (e.g. NaOH) treatment, acid treatment, plasma treatment, freezing/thawing.
A treated surface to increase the surface area is defined as a surface with increased surface roughness due to a chemical or physical treatment (e.g. base, acid, plasma, or freezing/thawing).
Covalently linkable molecules to facilitate a chemical cross-linking to the surface area are Molecules that have a functional group for attachment to the surface and another functional group that can be bound to other molecules. Examples are Aminopropyl methacrylamide (APMA), Gelatin methacrylate (GelMA), and N-hydroxy succinimide) ester diazrine.
Salts are defined as a chemical that contains positively charged and negatively charged ions. Examples include calcium chloride and calcium sulfate. To stimulate surface-initiated physical crosslinking, calcium chloride (CaCl2)) or calcium sulfate (CaSO4) was deposited on the surface of the implants. Other salts of divalent cations (e.g. Ca2+, Mg2+, Sr2+, Zn 2+(zinc2+)) or multivalent cations (e.g. Ti4+ or Al3+) could also be used for surface initiated physical crosslinking.
Hydrogel precursor solution is defined as a solution that contains a crosslinkable polymer and an initiator.
Charged polymers are defined as polymers that carry negative charge or positive charge including alginate, polyglutamic acid, etc.
Covalently reactive macromonomers are defined as polymer molecules that carry chemically reactive groups.
Initiators are defined as chemicals that initiate a chemical reaction. Examples include photoinitiators (e.g. Lithium phenyl-2,4,6-trimethylbenzoylphosphinate) and chemical initiators (e.g. Ammonium persulfate).
Biologics are defined as any molecules or organisms that can interact in living systems. Examples are proteins, peptides, cells, DNA, RNA, drugs, antibiotics.
Coating with one or more layers is defined as a single layer of coating with thickness in the 10-1000 μm range or multiple layers of coating each having thickness in the 10-1000 μm range.
Tissue Engineering is defined as engineering or regeneration of rigid or soft tissues including bone, cartilage, tendon, ligament, muscle, heart, heart valves, etc.

A facile method was developed for manufacturing PCL-TCP/hydrogel composite scaffolds, hereafter referred to as hybrid tissue engineering construct (HyTEC). After 3D printing, the surface of PCL-TCP scaffolds was treated in three steps to increase hydrophilicity, improve hydrogel adhesion and stimulate surface-initiated crosslinking. A physically crosslinked hydrogel was then loaded on the scaffolds followed by covalent crosslinking of the hydrogel to form a stable interpenetratable network. The effects of surface treatment, processing parameters and freeze-drying on hydrogel loading and preservation of porous structure of the scaffold after manufacturing were investigated. Further, the adhesion between the PCL-TCP and hydrogel as well as the release kinetics of BMP2 protein encapsulated in HyTEC were evaluated. Moreover, the biocompatibility and osteo-inductive potential of BMP2 loaded HyTEC were studied. The method/strategy could be used for incorporating a wide range of hydrogels into porous polyester-based scaffolds as well as coating non-porous polyester-based constructs. As an example, at the end of this description the inventors demonstrated the efficacy of the method/strategy for coating non-porous PCL-TCP rods by modifying hydrogel and crosslinking mechanism for sustained release of BMP2 protein and for cell encapsulation.

### Materials and Methods

The following description of materials and methods are exemplary embodiments.

### Materials

Medical-grade polycaprolactone (PCL, Mn=80 kDa) was purchased from Sigma-Aldrich. β-TCP nano-powder with average particle size of 100 nm (TCP) was received from Berkeley Advanced Materials Inc. Dimethylformamide (DMF), sodium hydroxide (NaOH) and ethanol were purchased from Fisher Scientific Inc. N-(3-Dimethylaminopropyl)-N′-ethylcarbodiimide hydrochloride (EDC), N-Hydroxysulfosuccinimide (NHS), 2-(N-Morpholino)ethanesulfonic acid (MES), N-(3-Aminopropyl)methacrylamide hydrochloride (APMA), Ammonium persulfate (APS), N,N,N′,N′-Tetramethylethylenediamine (TEMED), gelatin type A, Heparin, ninhydrin and Triton X-100 were purchased from Sigma-Aldrich. Ninhydrin reagent was prepared by dissolving 20 mg/mL ninhydrin in ethanol. Polyethylene dimethacrylate (PEGDMA, Mn=1000 gr/mol) was received from Polyscience, Inc. Sodium alginate (alginate, 500GM) was purchased from Pfaltz & Bauer Inc. Human BMP2 protein was provided by Medtronic. Calcium colorimetric assay (MAK022), CCK-8 kit and Human BMP2 ELISA kit were purchased from Sigma-Aldrich. QuantiChrom ALP Assay Kit was received from BioAssay Systems LLC. Quant-it PicoGreen assay kit was purchased from Thermo Fisher Scientific.

### Methods

### Synthesis of PCL-TCP Filament and 3D Printing

PCL-TCP filament with PCL to TCP weight ratio of 80:20 was synthesized as described by Bruyas (Bruyas et al., Effect of Electron Beam Sterilization on Three-Dimensional-Printed Polycaprolactone/Beta-Tricalcium Phosphate Scaffolds for Bone Tissue Engineering, Tissue Eng Pt A (2018)). Briefly, 80 gr of PCL and 20 gr of TCP were dissolved in 800 mL and 400 mL of DMF, respectively at 80° C. with continuous stirring for 3 hours. The PCL and TCP solutions were then mixed and stirred for one hour, followed by precipitation in 4 liters of water to make PCL-TCP composite. The PCL-TCP composite was rinsed with water to remove the residual solvent and air dried at ambient temperature for 24 hours. The dried PCL-TCP composite was cut into pellets and extruded using an in-house built screw extruder as described by Bruyas (Bruyas et al, Systematic characterization of 3D-printed PCL/beta-TCP scaffolds for biomedical devices and bone tissue engineering: Influence of composition and porosity, J Mater Res 33(14) (2018) 1948-1959). PCL-TCP scaffolds were 3D printed using a Lulzbot Mini (Aleph Objects Inc, USA) with a nozzle diameter of 500 μm. For surface characterization, non-porous disks with 10 mm diameter and 600 μm thickness and for all other tests porous cylinders with 10 mm diameter and 5 mm height were printed. The 3D models were designed using SolidWorks (SolidWorks Corp.) and sliced using Cura software. For printing 0%, 30%, 50%, 60%, 70% and 80% porous scaffolds, strut distances of 0.4, 0.53, 0.80, 1.00, 1.25 and 2.00 mm were used. The printing temperature, layer thickness and printing speed were set to 160° C., 200 μm and 5 mm/s, respectively as described by Bruyas (Bruyas et al., Effect of Electron Beam Sterilization on Three-Dimensional-Printed Polycaprolactone/Beta-Tricalcium Phosphate Scaffolds for Bone Tissue Engineering, Tissue Eng Pt A (2018). To synthesize methacrylated gelatin (GelMA) macromonomer, gelatin was dissolved in DI water (10% w/v) at 50° C. Methacrylic anhydride was added to gelatin solution at a molar ratio of 100:1 (methacrylic anhydride:gelatin) and the solution was allowed to react under stirring for 1 hr at 50° C. The mixture was then 5× diluted with DI water and dialyzed against DI water using a dialysis tube (Spectrum Laboratories, Rancho Dominquez, CA) with 6-8 kDa molecular weight cutoff for 3 days at 40° C. The GelMA solution was then freeze-dried and stored at −80° C.

To synthesize methacrylated heparin (HepMA), 1 gr heparin was dissolved in 100 mL MES buffer (100 mM). 5 mL MES buffer containing 45 mg EDC and 30 mg NHS was then added to the heparin solution to activate the carboxylic acid groups as described by Jeon (Jeon et al, Affinity-based growth factor delivery using biodegradable, photocrosslinked heparin-alginate hydrogels, J Control Release 154(3) (2011) 258-66). After 1 hr reaction at room temperature, 25 mg APMA in 1 mL MES was added to the solution and allowed to react for 2 hr at room temperature. The methacrylated heparin solution was then dialyzed against DI water using a dialysis tube (Spectrum Laboratories, Rancho Dominquez, CA) with 6-8 kDa molecular weight cutoff for 3 days at ambient temperature, lyophilized, and stored at −80° C.

### Hydrogel Loading on 3D Printed Porous Scaffolds

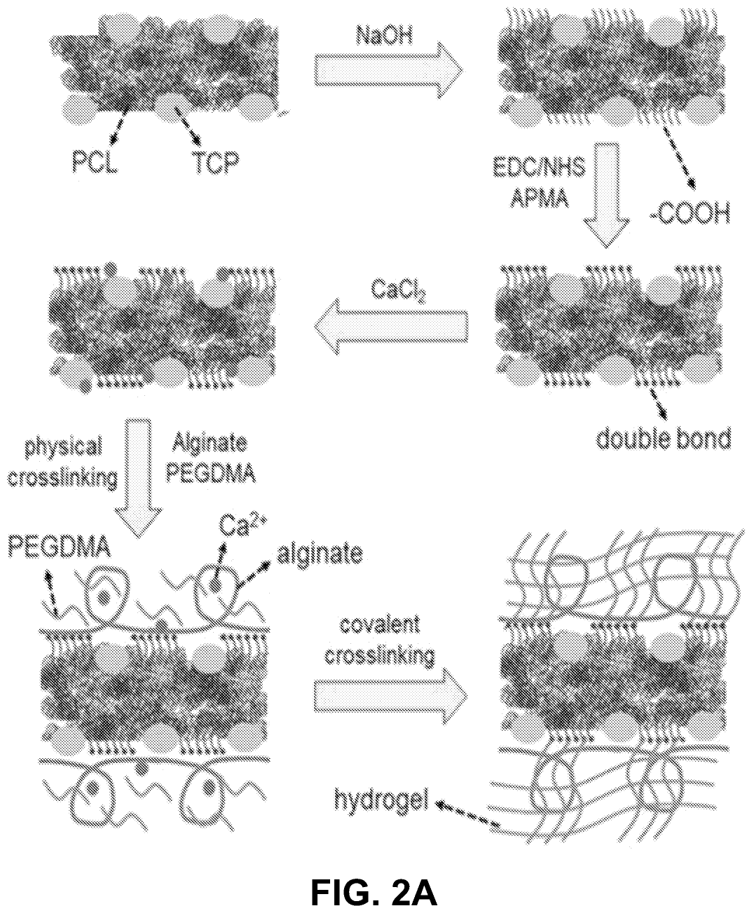

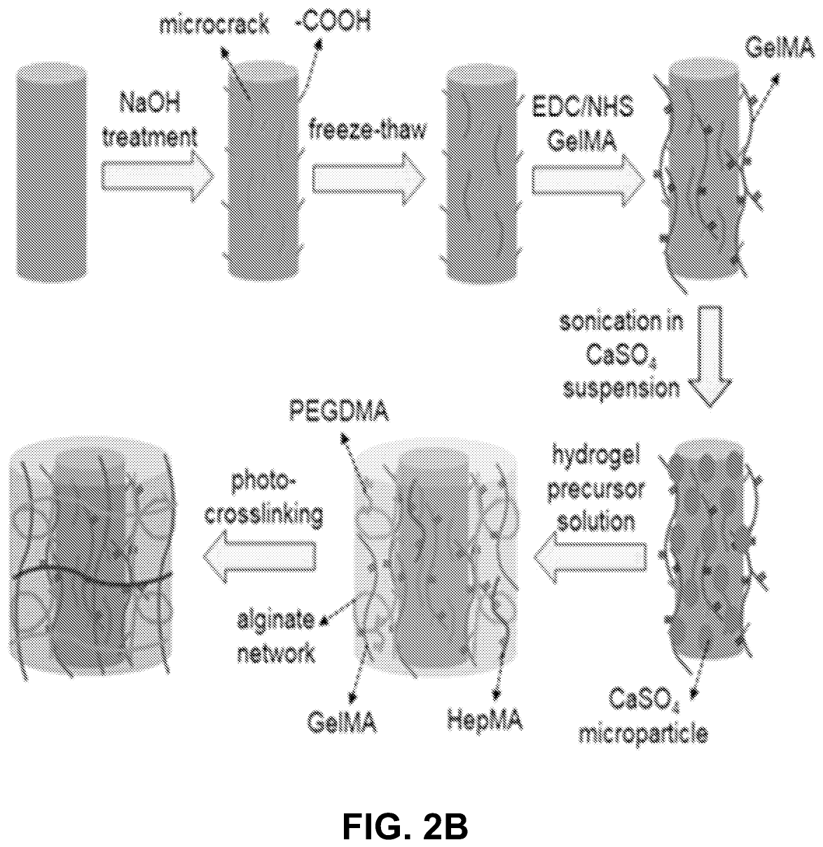

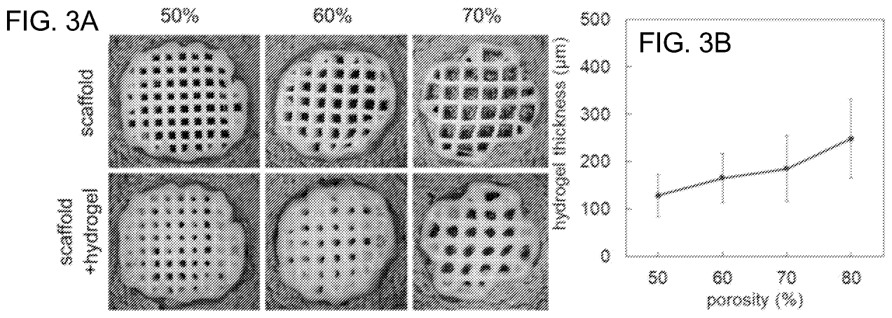

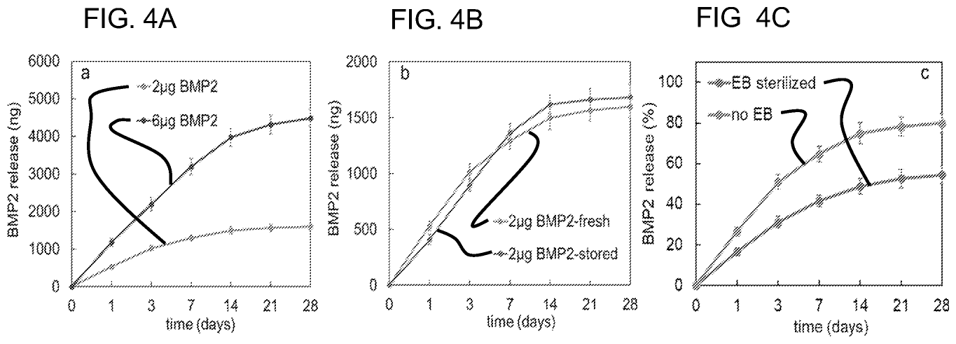

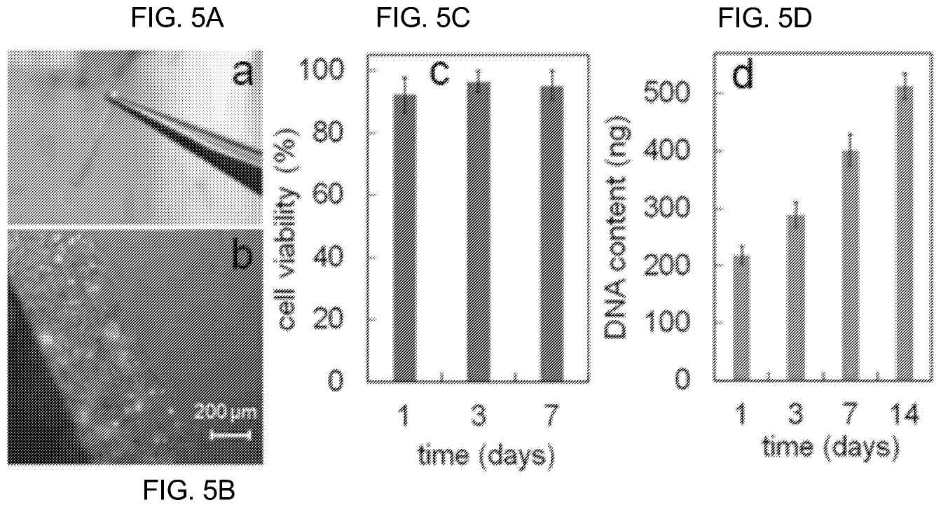

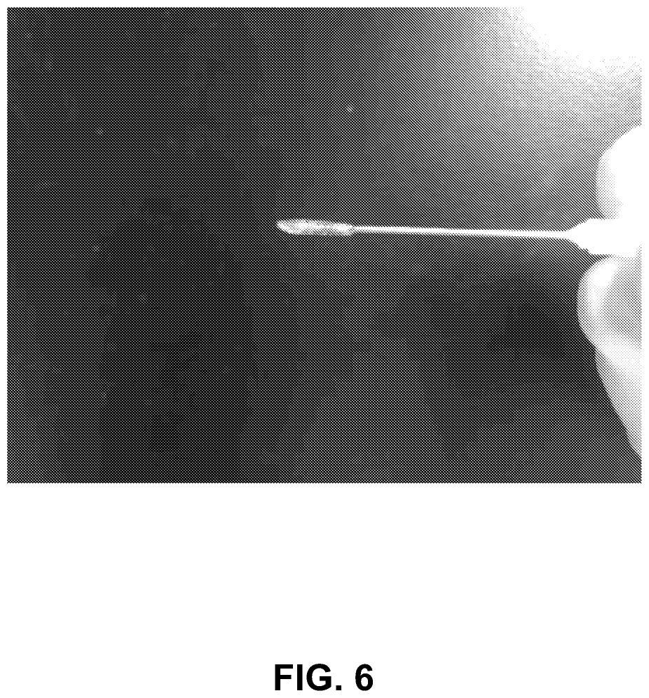

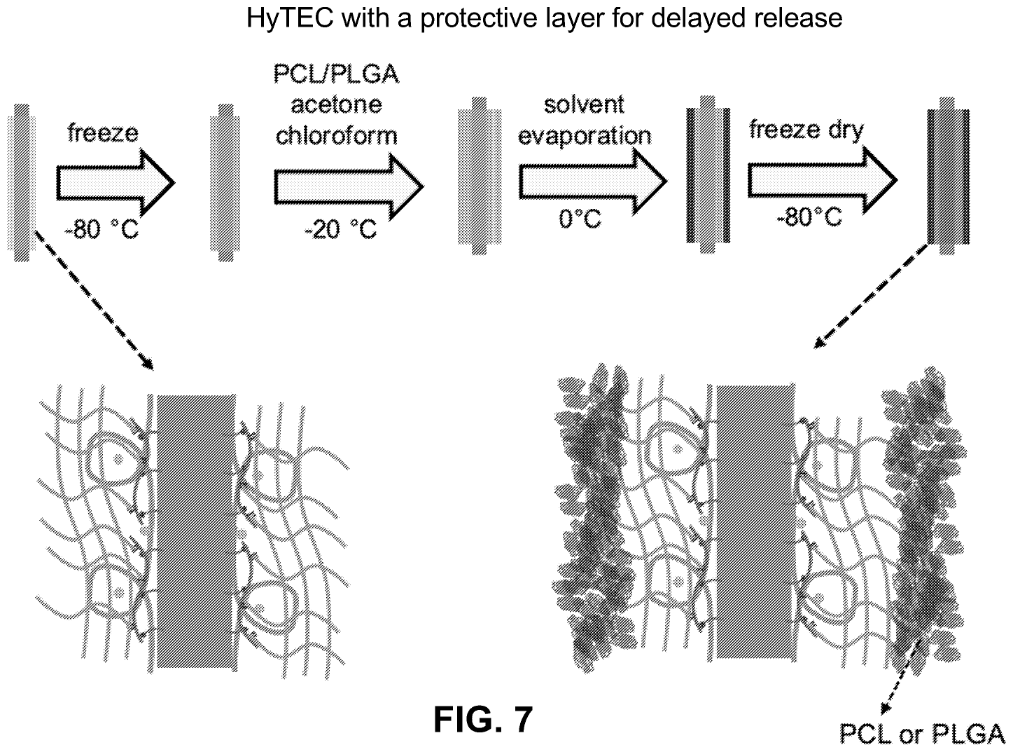

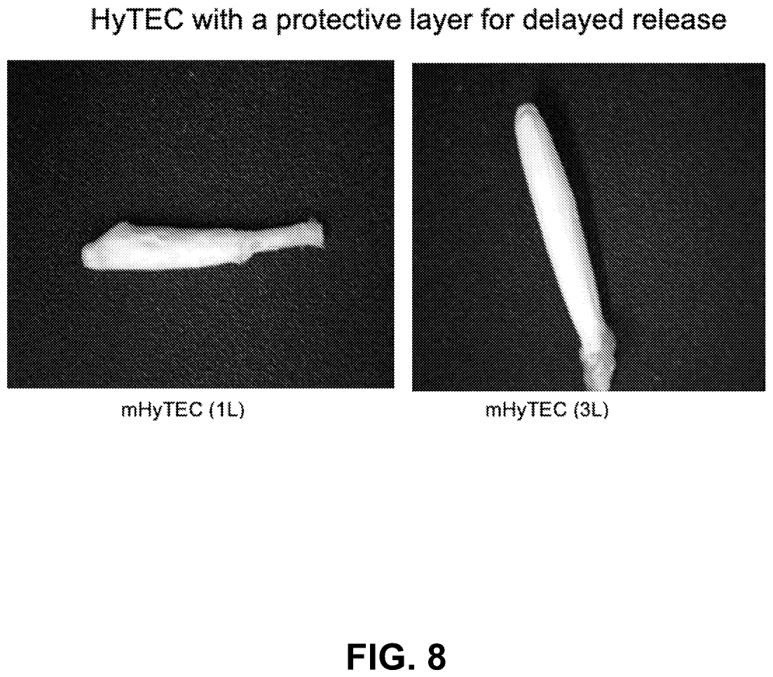

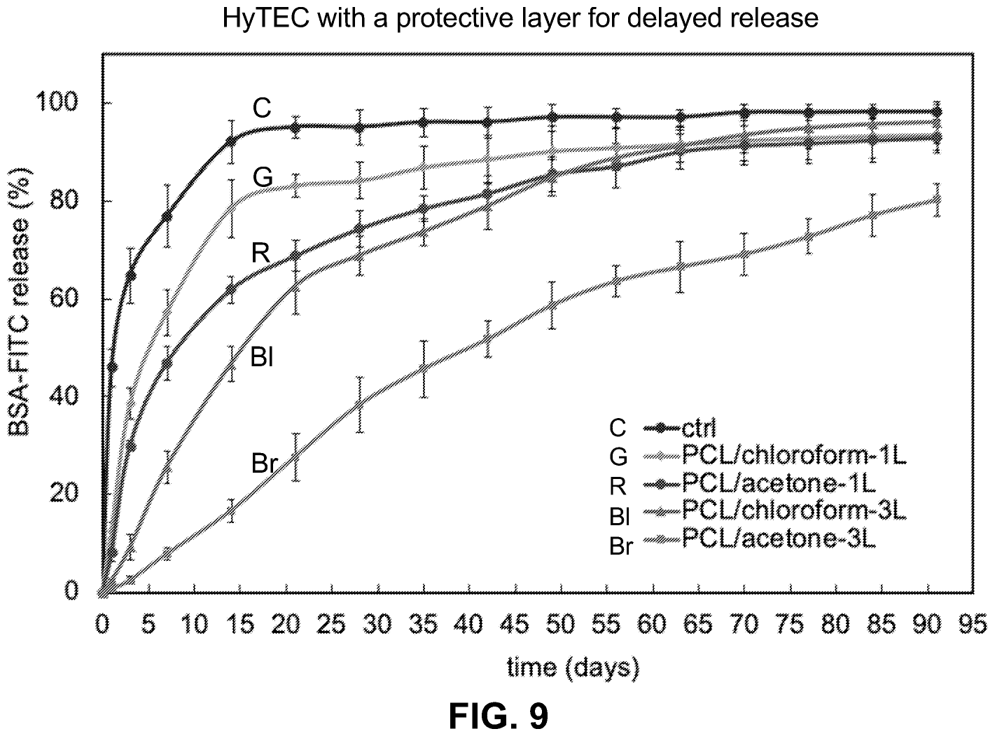

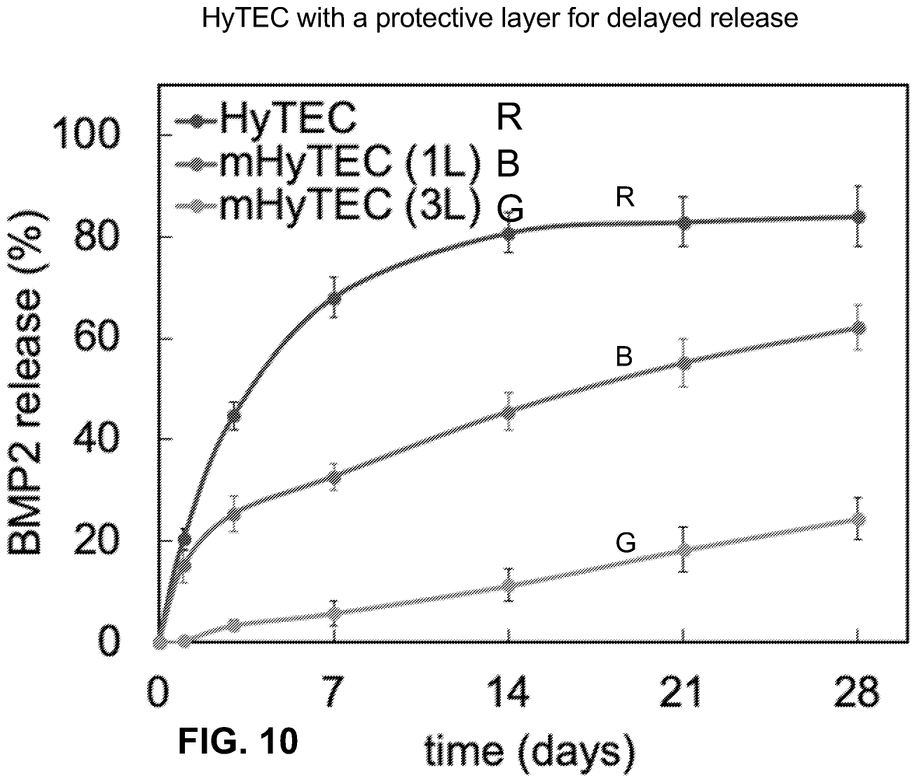

The procedure for scaffold surface treatment and hydrogel formation is shown schematically in FIG. 2A. The 3D printed scaffolds were dipped into a 5N NaOH solution and centrifuged at 1000 rpm for 1 min to ensure penetration of the NaOH solution into the pores of the scaffolds. The scaffolds were incubated in the NaOH solution for an hour unless otherwise specified and then washed 3 times with DI water. The scaffolds were then incubated in an MES buffer (100 mM) containing EDC (5 mg/mL) and NHS (5 mg/mL) for 30 min at room temperature to activate the carboxylic acid groups on the surface. The scaffolds were then treated with APMA (0.25, 2.5 or 10 mg/mL) in MES (100 mM) buffer for 30 min at room temperature and washed with DI water. The surface of the APMA treated scaffolds was wetted with CaCl2) solution (20-200 mg/mL in DI water) through incubation of the scaffold in CaCl2) solution for 1 hour at room temperature and then centrifugation of the scaffolds at 1000 rpm for 1 min to remove the residual solution. The surface modified CaCl2) treated scaffolds were vacuum dried for 3 hours and used for hydrogel loading.

The hydrogel precursor solution was prepared by dissolving PEGDMA (10-30% wt/vol), alginate (1.5% wt/vol) and 1 mg/mL APS in DI water. The surface treated scaffolds were dipped into the hydrogel precursor solution for 1 min at room temperature and then centrifuged at 1000 rpm for 1 min to remove the residual precursor solution. At this step, a layer of hydrogel was formed on the scaffold surface due to the diffusion of calcium from the surface and gelation of alginate. The hydrogel coated scaffolds were then incubated in APS (9 mg/mL) and TEMED (6 mg/mL) in DI water solution for 5 min to crosslink the PEGDMA macromonomers within the hydrogel layer and form an interpenetrating network. The scaffold/hydrogel composite was washed with DI water to remove the residual initiator or unreacted macromonomers.

Coating Non-Porous Rods with a Bioresorbable Hydrogel

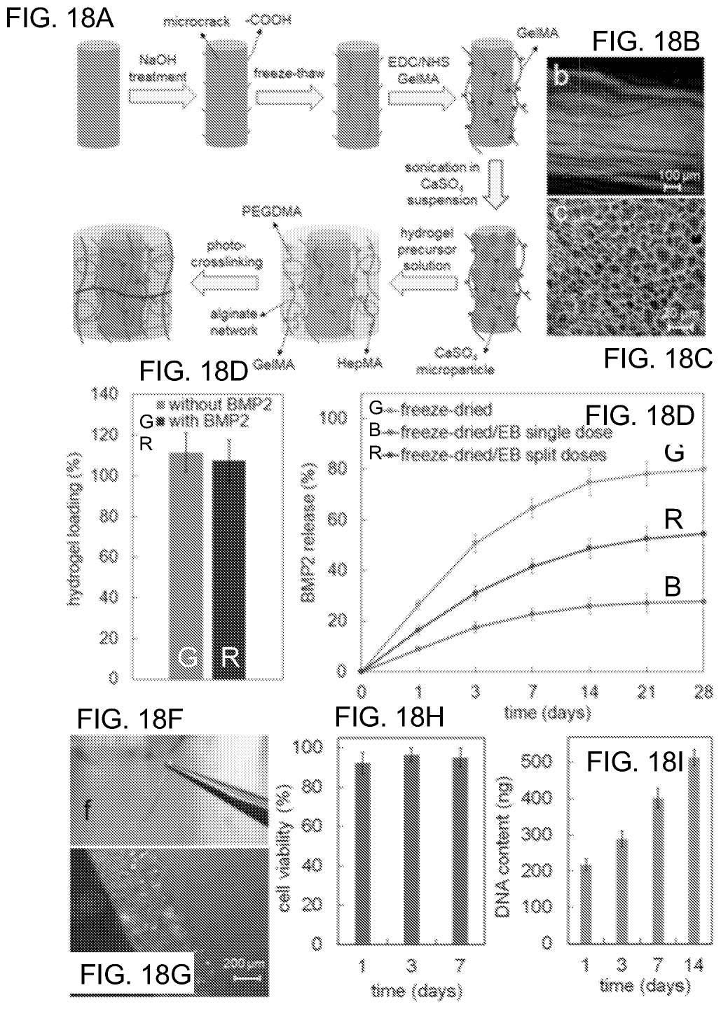

The procedure for coating non-porous PCL-TCP rods with a bioresorbable hydrogel is schematically shown in FIG. 18A. PCL-TCP filaments with 0.9 mm diameter were synthesized as described infra, manually cut to make 15 mm rods, and dipped into a 5N NaOH solution for 6 hours. The rods were then washed three times with DI water and incubated in an MES buffer (100 mM) containing EDC (5 mg/mL) and NHS (5 mg/mL) for 30 min at room temperature. Then, the rods were washed three times with DI water and incubated in gelatin methacrylate (GelMA) 2% solution in MES buffer for 1 hour at 37° C. The rods were then washed three times with DI water to remove the unreacted GelMA and incubated in EDC/NHS (5 mg/mL) in MES buffer solution for 15 minutes at room temperature. The GelMA coated rods were then washed three times with DI water and dried under vacuum. Then, the GelMA coated rods were dipped into a CaSO4 suspension in DI water (10-200 mg/mL) at 60° C. and sonicated for 30 seconds. The rods were then transferred into wells of a 24-well plate and dried under vacuum. The dried rods were dipped into wells of a 96-well plate containing GelMA (15%), Alginate (1.25%), PEGDMA (2%), HepMA (1%), protein (BMP2, 200 μg/mL), and photoinitiator (0.3%) in DI water at 37° C. for 2 minutes. The hydrogel-loaded rods were removed from the solution and left in dry wells of a 96-well plate for 5 minutes. The hydrogel-loaded rods were then irradiated with visible light for 15 minutes to covalently crosslink GelMA, PEGDMA, and HepMA. The crosslinked hydrogel-loaded rods were stored at −80° C. and freeze-dried.

### Freeze Drying and E-Beam Sterilization

For freeze drying, the HyTECs were submerged in liquid nitrogen for 30 min and then lyophilized and stored at 4° C. For rehydration, the freeze-dried HyTECs were incubated in DI water for 15 min before further analysis. For E-beam sterilization, HyTECs were exposed to E-beam irradiation at a standard single dose of 25 kGy, following norm ISO 11137-2:2006. E-beam sterilization of BMP2-laden hydrogel-loaded PCL-TCP rods was performed with a single dose (25 kGy) or two (12.5 kGy) doses to see the effect of splitting E-beam dose on the activity of loaded protein.

### Surface and Mechanical Characterization

The density of grafted APMA on PCL-TCP scaffolds was quantified via measuring the concentration of unreacted APMA in the solution after the reaction using ninhydrin assay. Briefly, the APMA solution after the reaction with PCL-TCP scaffold was diluted 10 times in MES buffer. 40 μL of the ninhydrin reagent was added to 200 μL of the diluted APMA solution. After mixing, the solution was heated to 90° C. in a capped tube for 8 min and the absorbance was read at 570 nm using a SpectraMax M2 plate reader (Molecular Devices LLC). The concentration of unreacted APMA in the solution was calculated using a calibration curve made for the absorbance of solutions with known concentrations of APMA. To evaluate the surface hydrophilicity of PCL-TCP constructs, a 4 μL water droplet was deposited on the disc scaffolds, the contact angle was measured using a goniometer Ramé-Hart 290 (Ramé-Hart instrument co., USA) and analyzed using image processing.

The apparent Young's modulus and the stress at yield of the scaffolds were tested using an Instron 5944 uniaxial testing system (Instron Corporation, Norwood, MA) with a 2 kN load-cell, a preload of 1N and a displacement rate of 1% strain/s. The initial slope of the stress vs strain curve was taken as the Young's modulus. The stress at yield was defined as the stress at which a line starting from 1% strain offset with a slope equal to the Young's modulus intersected with the stress vs strain curve.

To measure the release of calcium ions from the surface of the scaffolds, the CaCl₂ treated scaffolds (or CaSO₄ treated non-porous rods) were incubated in 1 mL DI water at room temperature for 1 hr. The concentration of Ca²⁺ ions in the release medium was measured using calcium colorimetric assay (MAK022, Sigma-Aldrich, USA) on a SpectraMax M2 plate reader (Molecular Devices LLC) at 575 nm.

To measure the hydrogel layer thickness, the scaffolds were imaged before and after hydrogel loading using a Dino-Lite digital microscope camera. The images were then analyzed using ImageJ to quantify the average hydrogel thickness. The average thickness of the gel in the quadrilateral pores was defined as half of the difference between the size of the empty spot (black color in images) before and after the hydrogel loading. The fraction of filled pores was defined as the ratio of the number of those quadrilateral pores that were completely filled with hydrogel to all quadrilateral pores.

The hydrogel loading (%) was calculated from the scaffold weight before hydrogel loading (Wb) and after hydrogel loading (Wa), using the following equation;

$$hydrogel loadng = 100 \times ( Wa - Wb ) / Wb$$

For scanning electron microscopy (SEM) imaging, the HyTEC samples were immersed in liquid nitrogen and freeze-dried. The freeze-dried samples were dipped in liquid nitrogen and cut using a surgical blade. The hydrogel samples were then coated with gold using a SPI sputter (SPI Supplier Division of Structure Prob, Inc., West Chester, PA) for 180 seconds and imaged using a Field Emission Scanning Electron Microscope (Zeiss Sigma, White Plains, NY) at an accelerating voltage of 5 keV.

### Hydrogel Interfacial Stiffness

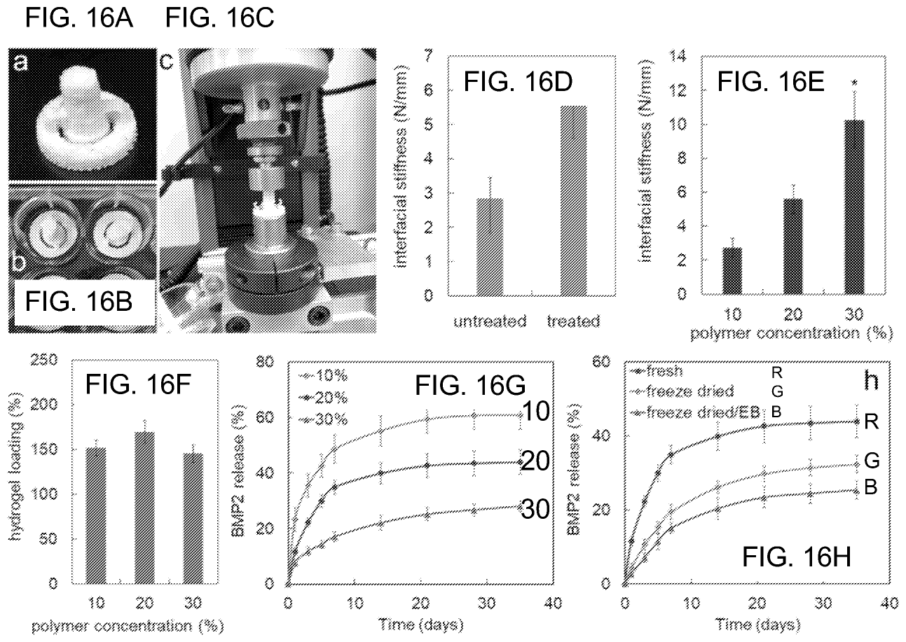

A customized 3D printed PCL-TCP device was designed and used to evaluate the adhesion of the hydrogels to PCL-TCP scaffolds (see FIGS. 16A-B). The device was composed of two concentric cylinders separated with a 400 μm gap and connected through two bridges. The hydrogel was made within the gap between two concentric cylinders. The hydrogel-incorporated device was then placed on an Instron 5944 uniaxial testing system (Instron Corporation, Norwood, MA) with a 100N load-cell and the bridges connecting inner and outer cylinders were cut (see FIG. 16C). The force needed to push the inner cylinder out of the device (interfacial stiffness) was measured via push-out tests using a 0.1N preload and 0.1 mm/s displacement rate.

### Protein Release

For measurement of release kinetics from porous HyTECs, BMP2 protein was added to the PEGDMA (10-30% wt/vol), alginate (1.5% wt/vol) and APS (1 mg/mL) precursor solution prior to hydrogel loading on 80% porous PCL-TCP scaffolds. The average hydrogel loading (relative to the scaffold weight) changed from 151% to 169% and 144% with increasing the PEGDMA concentration from 10% to 20% and 30% (see FIG. 16F). To load 1.5 μg BMP2 onto all scaffolds, 9.0, 8.0 and 9.5 μg/mL BMP2 was added to 10%, 20% and 30% PEGDMA precursor solutions, respectively. The BMP2-laden HyTECs were incubated in 1 mL PBS at 37° C. for 35 days. At each time point, the amount of BMP2 in the release medium was measured using ELISA and the release medium was replaced with 1 mL of fresh PBS.

For measurement of BMP2 release kinetics from non-porous HyTECs, the rod-shaped HyTECs with 2 μg encapsulated BMP2 were freeze-dried and incubated in 1 mL PBS at 37° C. for 28 days. At each time point, the amount of BMP2 in the release medium was measured using ELISA and the release medium was replaced with fresh PBS.

### Cell Culture

Human Mesenchymal Stem Cells (hMSCs) and Multi-potent mouse C3H10T1/2 fibroblasts (ATCC, USA) were cultured in DMEM medium (Life Technologies, USA) supplemented with 10% fetal bovine serum (FBS, Life Technologies, USA) and 1% Penicillin and Streptomycin (hereafter referred to as culture medium) at 37° C. in a 5% CO2 humidified incubator. After reaching 70% confluency, hMSCs or C3H10s were enzymatically lifted with trypsin-EDTA and used for in-vitro studies. All cells were passaged <6 times prior to the in-vitro studies.

### Biocompatibility of Porous HyTECs

For in-vitro cell studies, the 80% porous PCL-TCP scaffolds after APMA surface modification and before CaCl2) treatment, were sterilized in 70% ethanol solution for 20 min.

The CaCl2) solution and hydrogel precursor solution were sterilized by filtration using 0.22 μm Millex syringe filters.

The scaffolds with or without hydrogel loading were incubated in 1 mL culture medium at 37° C. The culture medium with no scaffold exposure incubated at 37° C. was used as the control group. At days 1 (for viability and proliferation tests) and 4 (for proliferation test) the incubated cell culture medium (hereafter referred to as conditioned medium) was used for viability and proliferation tests.

For cell viability test, concurrent with incubation of scaffold or scaffold/hydrogel in culture medium, hMSC and C3H10 cells were seeded on 96 well plates at 5000 cells/well and incubated at 37° C. and 5% CO2 for 24 hr. The cultured medium was then replaced with 100 μL of the conditioned culture medium and the cells were incubated for another 24 hr. To measure the cell viability, 10 μL of the CCK-8 solution (CCK-8 kit, Sigma-Aldrich) was added to each well and after 3 hours of incubation, the absorbance was read at 450 nm on a plate reader. The viability of cells in the experimental groups (scaffold, scaffold+gel) was divided by that of cells in the control group (no scaffold) to calculate the normalized viability.

For cell proliferation test, concurrent with incubation of scaffold or scaffold/hydrogel in culture medium, hMSC and C3H10 cells were seeded on 24 well plates at 10000 cells/well and incubated at 37° C. and 5% CO2 for 24 hr. The cultured medium was then replaced with 600 μL the conditioned culture medium and the cells were incubated for 7 days. 3 days after addition of conditioned medium (4 days after cell seeding), the medium was replaced with fresh conditioned medium. At days 0, 3 and 7, cells were washed with PBS, enzymatically lifted using 250 μL of 0.25% trypsin-EDTA solution (Life Technologies, USA) and counted using a Z2 particle counter (Beckman Coulter, USA).

### Osteo-Inductive Potential of BMP2-Laden Porous HyTECs

The HyTECs without loaded BMP2 (scaffold+gel) or with 1.5 μg loaded BMP2 (scaffold+gel/BMP2), were incubated in 1 mL culture medium at 37° C. The conditioned medium was used for cell differentiation test every 3 days and replaced with fresh culture medium. The culture medium with no scaffold and no BMP2 (ctrl), culture medium supplemented with 1.5 μg/mL BMP2 (BMP2 in medium (3 d)) and culture medium supplemented with 214 ng/mL BMP2 for 21 days (BMP2 in medium (21 d)) incubated at 37° C. were used as control groups. Concurrent with incubation of experimental and control groups in culture medium, hMSC and C3H10 cells were seeded on 24 well plates at 10000 cells/well and incubated at 37° C. and 5% CO2 for 24 hr. The cultured medium was then replaced with 600 μL of the conditioned culture medium and the cells were incubated for 21 days with changing the medium to fresh conditioned medium every 3 days. For BMP2 in medium (3 d) group, the medium was changed to culture medium (BMP2 free) after 3 days. At each time point (day 0, 7, 14 and 21), cells were washed with PBS and lysed with 1% Triton X-100 in PBS using a cell scraper followed by shaking for 20 min at room temperature. The lysate was centrifuged at 2000×g for 15 min at 4° C. and the supernatant was collected. The ALP activity in the supernatant was measured using QuantiChrom ALP Assay Kit (BioAssay Systems, Hayward, CA, USA) according to the manufacturer's Instructions, on the plate reader at 405 nm. The double-stranded DNA content of the lysate was measured using PicoGreen assay kit (Quant-it, Thermo Fisher Scientific). The ALP activity was divided by the DNA content to calculate the normalized ALP activity.

### Cell-Laden HyTECs

PCL-TCP filaments with 0.9 mm diameter were synthesized and coated with GelMA as described in the previous section. Then, the GelMA coated rods were dipped into a CaSO4 suspension in DI water (100 mg/mL) at 60° C. and sonicated for 30 seconds. The rods were then transferred into wells of a 24-well plate, dried under vacuum, and sterilized under UV for 60 minutes. hMSCs were suspended in hydrogel precursor solution containing GelMA (10%), Alginate (1.25%), PEGDMA (2%), and photoinitiator (0.3%) in calcium-free culture medium at 2 million cells/mL density. The sterile rods were then dipped in the cellular precursor solution for 2 minutes at 37° C. The cell-laden hydrogel-loaded rods were removed from the solution, left in sterile dry wells of a 96-well plate for 5 minutes, and then irradiated with visible light for 15 minutes. The cell-laden hydrogel coated rods were then transferred into wells of a 24-well plate and incubated in culture medium at 37° C. and 5% CO₂.

For live/dead cell imaging, cell-laden HyTECs were stained with Calcein AM (2 μM) and Ethidium homodimer-1 (4 μM) according to manufacturer's instructions and imaged using a Zeiss AxioObserver Z1 fluorescent microscope. The live/dead images were divided into smaller squares and the number of live and dead cells were counted manually to calculate the cell viability. To quantify the DNA content of the HyTECs, at each time point, the samples were transferred into new wells and incubated in 500 μL of DMEM medium supplemented with collagenase (1 mg/mL) for 1 hour at 37° C. Then, 250 μL of triton solution (3%) in PBS was added to each well and the attached cells were scrapped from the surface using a CytoOne cell scraper (USA Scientific Inc, Ocala, FL). Then the cell suspension was transferred to a microcentrifuge tube and sonicated. The cell lysate was then centrifuged at 2000×g at 4° C. for 15 min and the supernatant was collected. The content of double-stranded DNA in the supernatant was measured using Quant-iT PicoGreen DNA assay according to manufacturer's instructions.

### Statistical Analysis

All experiments were done in triplicate. Statistically significant differences between groups were tested using a two-way ANOVA with replication, followed by a two-tailed Students t-test. A p-value smaller than 0.05 (p<0.05) was considered statistically significant.

### Results

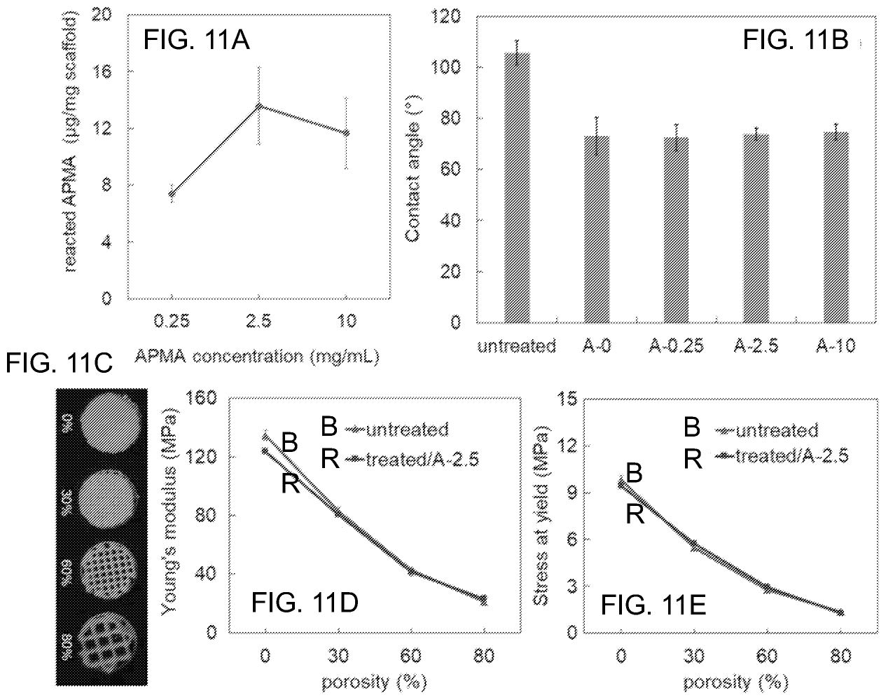

The density of grafted APMA on PCL-TCP constructs versus APMA concentration in the reaction solution is shown in FIG. 11A. The density of grafted APMA increased from 7.4 to 13.6 (μg/mg scaffold) when the APMA concentration increased from 0.25 to 2.5 mg/mL. The density of grafted APMA did not significantly change with increasing the APMA concentration from 2.5 to 10 mg/mL. The effect of surface modification with APMA on the contact angle of PCL-TCP constructs is shown in FIG. 11B. The contact angle of PCL-TCP constructs without NaOH treatment (untreated) was 105.6°, indicating a hydrophobic surface. The contact angle of constructs decreased to 73.1°, after NaOH treatment (A—0). That shows the PCL-TCP surface was relatively hydrophilic after NaOH treatment due to the scission of PCL ester groups to hydroxyl and carboxyl groups. The contact angle, hence hydrophilicity of PCL-TCP constructs did not significantly change with incorporation of APMA at 0.25 (A—0.25), 2.5 (A—2.5) or 10 (A—10) mg/mL concentration. The 3D printed porous scaffolds were treated with 2.5 mg/mL APMA solution unless otherwise specified. PCL-TCP scaffolds with porosity ranged from 0% to 80% (FIG. 11C) were fabricated to investigate the effect of surface treatment on the mechanical properties of scaffolds. The Young's modulus and stress at yield of PCL-TCP scaffolds without surface treatment (untreated, B) and with NaOH treatment followed by APMA grafting (treated/A—2.5, R) is shown in FIGS. 11D-E, respectively. The Young's modulus of untreated scaffolds decreased from 134.7 to 83.0, 42.5 and 21.2 (MPa) when the porosity increased from 0% to 30%, 60% and 80%, respectively. The surface modification reduced the Young's modulus of 0% porous scaffolds from 134.7 to 123.7 (MPa). There was no significant difference between the Young's modulus of untreated and treated scaffolds at 30%, 60% or 80% porosity. The stress at yield of untreated scaffolds ranged from 9.8 to 1.3 (MPa) when the scaffold porosity increased from 0% to 80%. The stress at yield of scaffolds did not significantly change with surface treatment, at any of the studied porosities.

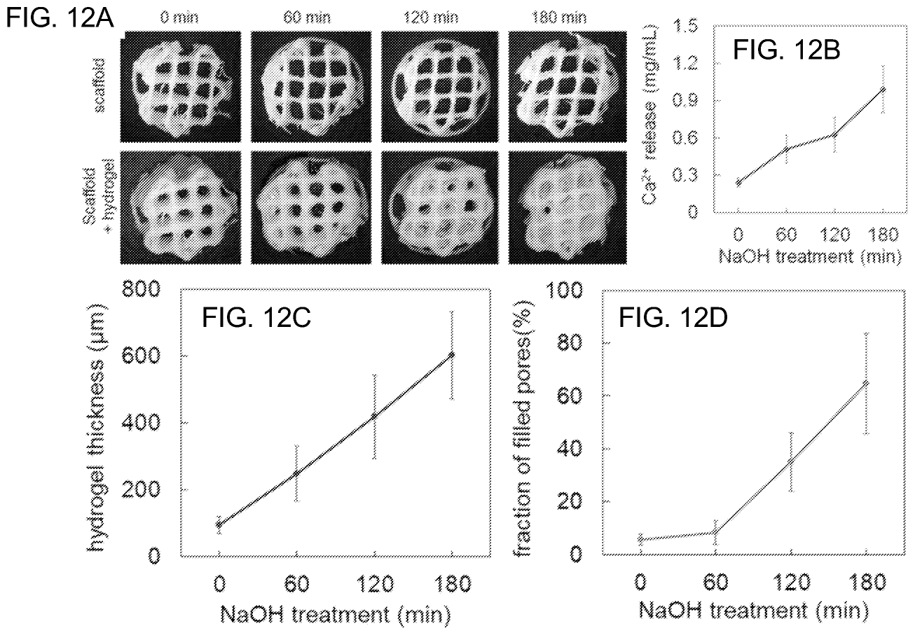

FIG. 12A shows the effect of NaOH treatment time on the hydrogel coating in 80% porous PCL-TCP scaffolds. The hydrogel layer thickness and fraction of filled pores are shown in FIG. 12A and FIG. 12D, respectively. The average hydrogel thickness increased from 94 to 248, 418 and 602 (μm) with increasing the NaOH treatment time from 0 to 60, 120 and 180 minutes. There was not a significant difference between the fraction of filled pores of untreated scaffolds (0 min) and those treated for 60 minutes in NaOH solution. The fraction of filled pores significantly increased when the NaOH treatment time increased from 60 to 120 and 180 minutes (see FIG. 12D). The effect of NaOH treatment time on the release of calcium ion from the surface of scaffolds is shown in FIG. 12B. The concentration of calcium in the release medium increased monotonically from 0.24 to 0.98 (mg/mL) with increasing the NaOH treatment time from 0 to 180 minutes. Therefore, an increase in the hydrogel thickness and the fraction of filled pores with extending the NaOH treatment time might be partially due to a larger released calcium from scaffolds.

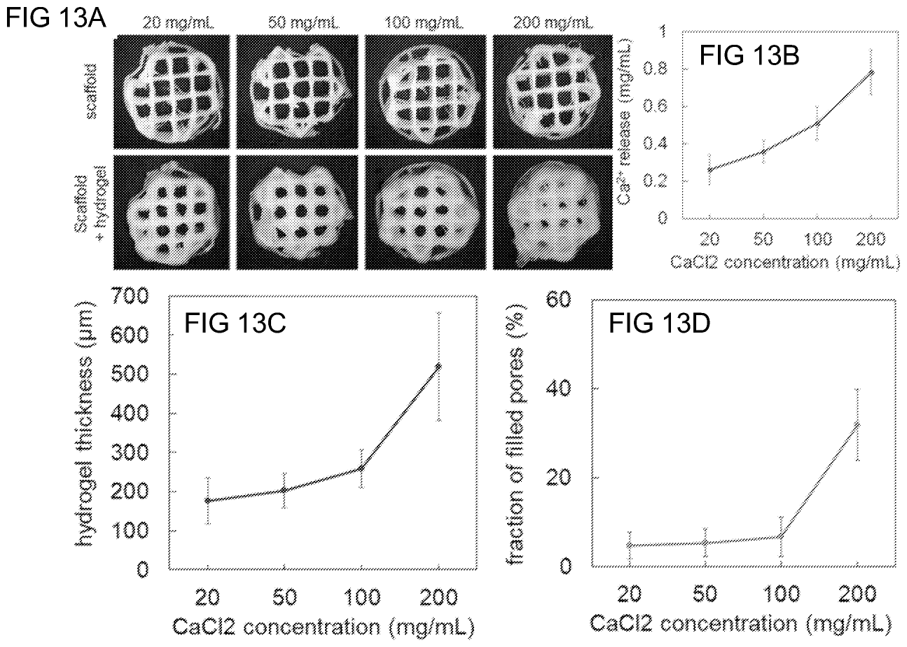

The effect of CaCl₂) concentration in the incubation solution on the hydrogel coating of 80% porous PCL-TCP scaffolds is shown in FIG. 13A. The hydrogel layer thickness and fraction of filled pores are shown in FIGS. 13C-D, respectively. The average hydrogel thickness and fraction of filled pores were below 259 μm and 6.7%, respectively, with no statistically significant change, when the CaCl₂ concentration ranged from 20 to 100 mg/mL. The average hydrogel thickness and fraction of filled pores increased from 259 μm to 519 μm and from 6.7% to 31.8%, respectively, with increasing the CaCl2) concentration from 100 to 200 mg/mL. The effect of CaCl₂) concentration in the incubation solution on the release of calcium ion from the surface of scaffolds is shown in FIG. 13B. The concentration of released Ca²⁺ in the release medium increased by 3 folds when the CaCl2) concentration in the incubation solution increased from 20 to 200 mg/mL. A dramatic change in the hydrogel thickness and the fraction of filled pores with increasing the CaCl₂ concentration from 100 to 200 mg/mL (see FIGS. 13B-C) could be due to a significant increase in the Ca2+ release (see FIG. 13D).

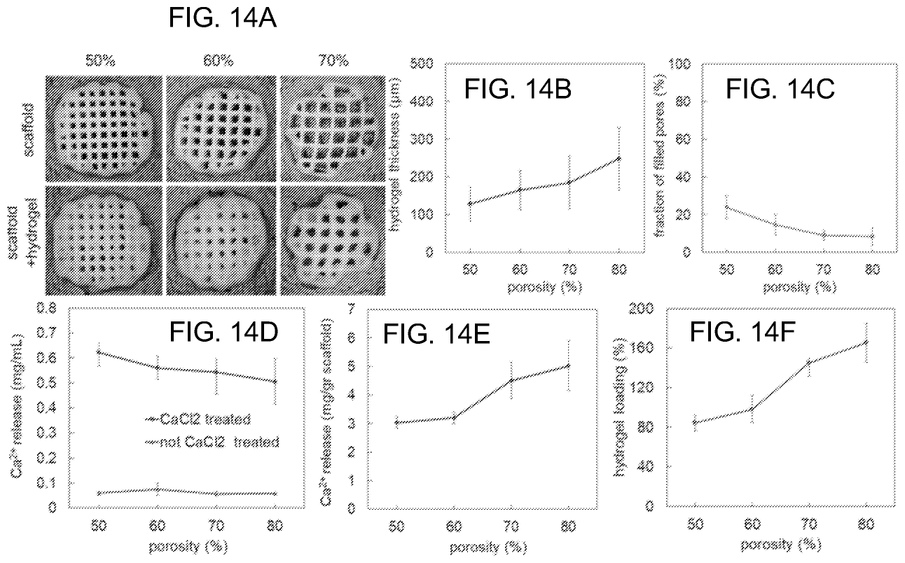

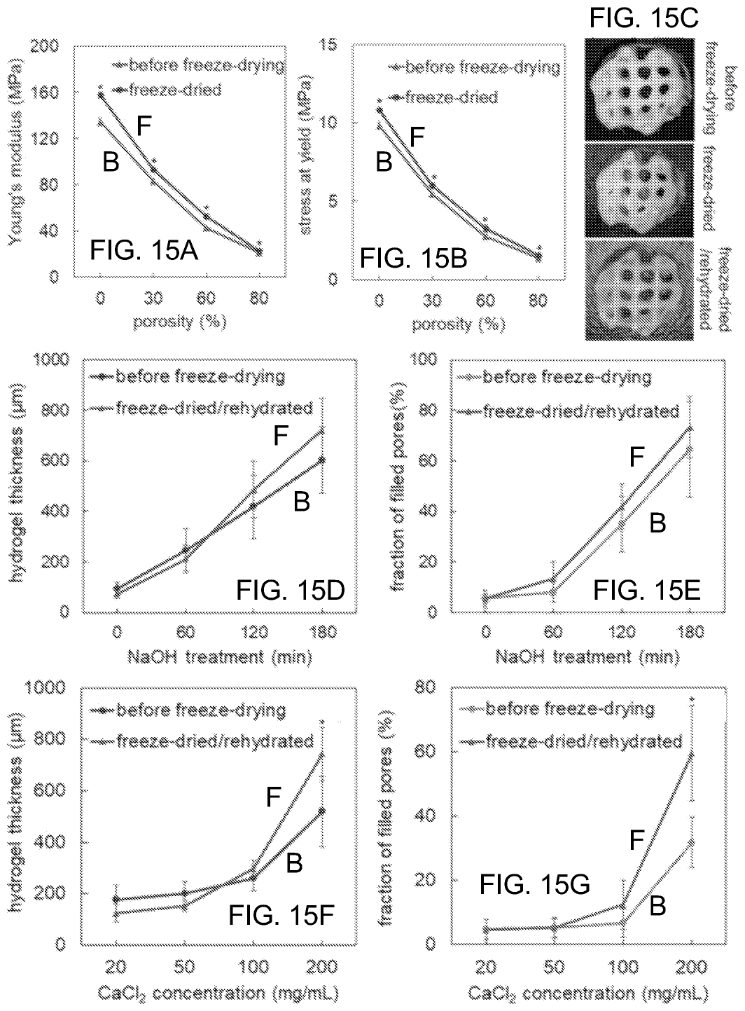

3D printed porous PCL-TCP scaffolds with 50%, 60% and 70% porosity, before and after hydrogel coating are shown in FIG. 14A. The effect of scaffold porosity on the hydrogel thickness and fraction of filled pores is shown in FIGS. 15B-C. The average hydrogel thickness increased from 128 to 248 μm while fraction of filled pores decreased from 23.9% to 8.4% with increasing the scaffold porosity from 50% to 80%. The effect of scaffold porosity on the release of calcium ion from the surface of scaffolds with incubation in CaCl₂ (100 mg/mL) solution (CaCl₂ treated, R) and without incubation in CaCl2 solution (not CaCl₂) treated, B) is shown in FIG. 14D. In the absence of CaCl₂) incubation, the average concentration of released Ca²⁺ in the release medium was below 0.07 mg/mL and did not significantly change with altering the scaffold porosity from 50% to 80%. The release of Ca²⁺ from scaffolds decreased from 0.62 to 0.50 mg/mL whereas the released Ca²⁺ per unit weight of scaffolds increased from 3.03 to 5.02 (mg/mg scaffold) when the porosity increased from 50% to 80% (FIGS. 14D-E). In addition, the hydrogel loading (relative to the scaffold weight) increased from 84% to 165%, when the porosity increased from 50% to 80% (FIG. 14F).

The effect of freeze drying on the mechanical properties of PCL-TCP scaffolds and characteristics of the hydrogel layer on the scaffolds are shown in FIGS. 15A-G. Freeze drying increased the Young's modulus and stress and yield of PCL-TCP scaffolds at all porosities (FIGS. 15A-B). The hydrogel layer remained intact after freeze drying and rehydration (FIG. 15C). In addition, freeze drying and rehydration did not significantly affect the hydrogel thickness and fraction of filled pores regardless of NaOH treatment time (FIGS. 15D-E). The hydrogel layer thickness and fraction of filled pores did not significantly change with freeze drying/rehydration when the CaCl₂) concentration in the incubation medium was 100 mg/mL or smaller. The hydrogel thickness and fraction of filled pores of freeze dried/rehydrated scaffolds were significantly higher than those of non-freeze dried scaffolds when the CaCl₂ concentration in the incubation medium was 200 mg/mL.

The structure of the 3D printed PCL-TCP device which was used to measure the adhesion of hydrogels to scaffolds is shown in FIGS. 16A-B. The hydrogel was made within the gap between two concentric cylinders. The hydrogel-incorporated device was then placed on the Instron machine and two bridges connecting inner and outer cylinders were cut and the interfacial stiffness was measured via push-out tests (FIG. 16C). The interfacial stiffness of the hydrogel increased by 2 folds from 2.84 to 5.60 (N/mm) when the scaffold surface was treated with APMA (FIG. 16D). In addition, the interfacial stiffness of the hydrogel significantly increased from 2.72 to 5.60 and 10.24 (N/mm) with increasing the PEGDMA concentration in the hydrogel solution from 10% to 20% and 30%, respectively (FIG. 16E). In contrast to the interfacial stiffness, the hydrogel loading on the scaffolds did not significantly change with PEGDMA concentration (FIG. 16F). The effect of PEGDMA concentration on the release kinetics of enzymatically active BMP2 protein from porous HyTEC scaffolds is shown in FIG. 16G. Following an initial burst release, the BMP2 was released steadily from the hydrogel-laden scaffolds over 7 days and then at a lower rate from day 7 to 35. The total enzymatically active released BMP2 protein from HyTEC after 35 days increased from 28% to 44% and 61% with decreasing the PEGDMA concentration from 30% to 20% and 10%, respectively. The effect of freeze drying and electron beam sterilization on the release kinetics of BMP2 from PCL-TCP/hydrogel scaffolds are shown in FIG. 16H. The BMP2 release of freeze-dried scaffolds after 35 day was 26% lower than that of fresh scaffolds. In addition, the BMP2 release of freeze dried and electron beam irradiated scaffolds was 22% lower than that of non-irradiated freeze dried scaffolds after 35 days.

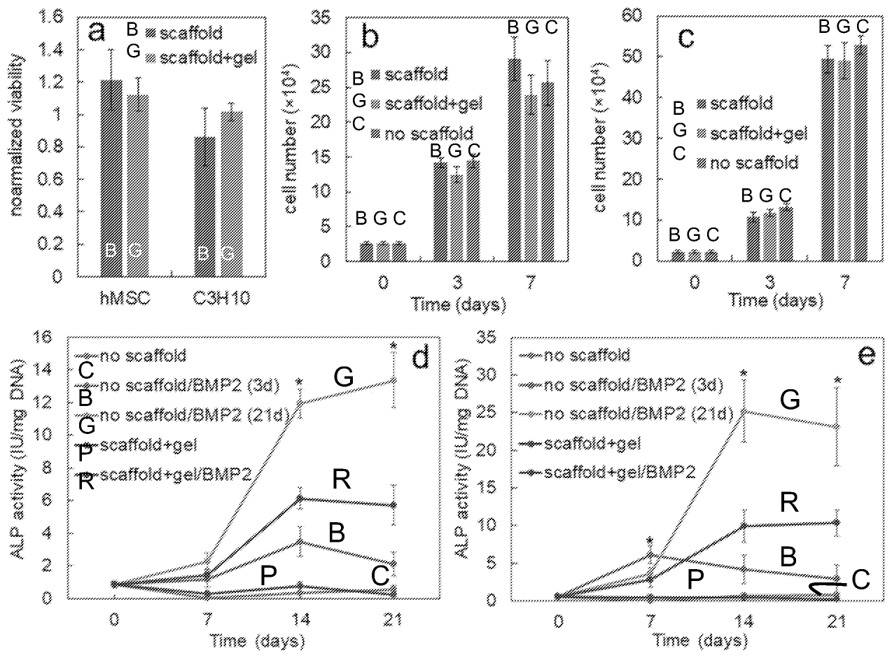

The normalized viability and proliferation of hMSC and C3H10 cells cultured in DMEM medium which was preconditioned with PCL-TCP/hydrogel or pristine PCL-TCP scaffolds are shown in FIGS. 17A-C. The viability of hMSCs or C3H10s cultured in scaffold/hydrogel preconditioned medium for 24 hours was not significantly different with the viability of cells cultured in scaffold preconditioned medium (FIG. 17A). The number of hMSCs in scaffold/hydrogel group increased from 2.6×104 at day 0 (before exposure to the preconditioned medium) to 12.5×104 and 24.0×104 at day 3 and 7, respectively (FIG. 17B). There was not a statistically significant difference between hMSC proliferation in scaffold/hydrogel or scaffold group with the control (no scaffold) group at any time point. The number of C3H10s in scaffold/hydrogel group increased from 2.2×104 at day 0 to 49.0×104 at day 7 (FIG. 17C). There was not a statistically significant difference between C3H10 proliferation in scaffold/hydrogel or scaffold group with the control (no scaffold) group at any time point. Therefore, the process of surface treatment and hydrogel formation did not adversely affect the cell viability and proliferation.

The ALP activity of hMSC and C3H10 cells cultured in DMEM medium (ctrl), DMEM medium preconditioned with scaffold/hydrogel without BMP2 (scaffold+gel), DMEM medium preconditioned with scaffold+BMP2 loaded hydrogel (scaffold+gel/BMP2), DMEM medium supplemented with 1.5 μg/mL BMP2 for 3 days (no scaffold/BMP2 (3 d)), and DMEM medium supplemented with 214 ng/mL BMP2 for 21 days (no scaffold/BMP2 (21 d)) are shown in FIGS. 17D-E. The ALP activity of hMSCs in ctrl and scaffold+gel group did not significantly increase in 21 days. The ALP activity of hMSCs in scaffold+gel/BMP2, no scaffolds/BMP2 (3 d) and no scaffold/BMP2 (21 d) groups increased significantly after 14 days of culture, then did not significantly increase from day 14 to 21. At days 14 and 21, the ALP activity of hMSCs in scaffold+gel/BMP2 group was significantly higher than that of no scaffolds/BMP2 (3 d) group and lower than that of no scaffolds/BMP2 (21 d) group. The ALP activity of C3H10s in ctrl and scaffold+gel group did not significantly change in 21 days of culture. The ALP activity of C3H10s in no scaffolds/BMP2 (3 d) group increased from day 0 to 7 and then decreased. The ALP activity of C3H10s in scaffold+gel/BMP2 and no scaffolds/BMP2 (3 d) groups rose significantly from day 0 to 14, then did not significantly change from day 14 to 21. At days 14 and 21, the ALP activity of C3H10s in scaffold+gel/BMP2 group was significantly higher than that of no scaffolds/BMP2 (3 d) group and lower than that of no scaffolds/BMP2 (21 d) group. Although the ALP activity of C3H10s in no scaffolds/BMP2 (3 d) group was significantly higher than that of scaffold+gel/BMP2 or no scaffolds/BMP2 (21 d) group at day 7, the peak ALP activity of no scaffolds/BMP2 (3 d) group over 21 days (6.1 IU/mg DNA), was lower than that of scaffold+gel/BMP2 (10.3 IU/mg DNA) or no scaffolds/BMP2 (21 d) (25.1 IU/mg DNA).

For making non-porous HyTEC constructs, NaOH treatment followed by freezing/thawing, GelMA conjugation to the surface, and CaSO₄ deposition were used to increase hydrophilicity, improve hydrogel adhesion and stimulate surface-initiated crosslinking, respectively. An SEM image of the surface of the hydrogel loaded on non-porous PCL-TCP rods is shown in FIGS. 18B-C. The surface of the hydrogel was porous with average pore size of 8 μm. The effects of CaSO4 concentration in the suspension, that was used for scaffolds treatment at 60° C., on the release of Ca2+ from the surface of PCL-TCP rods showed that there was no detectable calcium release when the rods without freezing/thawing were treated in 10 mg/mL or 20 mg/mL CaSO4 suspension. The amount of calcium release from the surface of the rods with or without freezing/thawing significantly increased with increasing the concentration of CaSO4 in the suspension from 50 to 200 mg/mL. Also, freezing/thawing significantly increased the amount of calcium release from the surface of the rods when the CaSO₄ concentration in the suspension was 20, 50, or 100 mg/mL. The effect of temperature of CaSO₄ suspension (50 mg/mL) during sonication on the calcium release from the surface of the rods showed that the calcium release significantly increased with increasing the CaSO₄ suspension temperature from 25° C. to 60° C. and did not change with further raising the temperature from 60° C. to 70° C. For the BMP2-laden hydrogel coating, the calcium deposition on the PCL-TCP rods was performed in a 50 mg/mL CaSO₄ suspension at 60° C. Average loading of hydrogel without BMP2 and with BMP2 on treated and calcium deposited PCL-TCP rods was 111% and 108%, respectively but there was not a significant difference between two groups (FIG. 18F). The release kinetics of BMP2 from freeze-dried BMP2-laden non-porous HyTEC over 28 days is shown in FIG. 18G (G). The amount of released BMP2 from the HyTECs increased in a sustained manner over 14 days and did not significantly change from day 14 to 28. Total amount of released BMP2 from freeze-dried HyTECs after 28 days was 80.0%. The release kinetics of BMP2 from non-porous HyTECs after freeze-drying and sterilization using single dose or split doses of E-beam is shown in FIG. 18FG (B and R). Total amount of released BMP2 from freeze-dried HyTECs after sterilization using single dose or split doses of E-beam was 27.8% and 54.5%, respectively after 28 days. FIGS. 18F-G show a cell-laden non-porous HyTEC and live/dead image of hMSCs in a HyTEC, respectively. The viability of hMSCs in HyTEC ranged between 92% and 96% over 7 days of incubation (FIG. 18H) while the DNA content of cellular HyTECs increased by 2.4 folds after 14 days of incubation (FIG. 18I).

### Considerations

The complications associated with autografts, allografts and DBM for treatment of large bone defects highlights the importance of developing synthetic bone grafts. A number of studies have shown PCL-TCP scaffolds are biocompatible, bioresorbable, mechanically stable and osteo-conductive. Further, owing to the low melting point and processability of PCL, PCL-TCP scaffolds with well-controlled porosity can be manufactured using Fused Deposition Modeling (FDM)-based 3D printing. However, lack of bone growth stimulating proteins limits the application of 3D printed PCL-TCP scaffolds for treatment of large bone defects. In the present invention, the inventors developed a postprocessing method for manufacturing interconnected porous, protein-laden thick hydrogel layer-coated 3D printed PCL-TCP scaffolds. Following 3D printing, the surface of scaffolds was treated in three consecutive steps to increase hydrophilicity, improve hydrogel adhesion and stimulate surface-initiated crosslinking. NaOH treatment imparts hydrophilicity to the surface of polyesters due to the scission of ester bonds to carboxyl and hydroxyl groups. Reactive double bonds were then incorporated onto the surface by grafting APMA to carboxyl groups using carbodiimide chemistry. To control the hydrogel layer thickness, the APMA modified scaffolds were treated with CaCl₂ solution to stimulate a surface-initiated crosslinking. When the CaCl₂ treated scaffolds were dipped into hydrogel precursor solution, the deposited CaCl₂) diffused from the surface to the solution, crosslinked alginate at the proximity of the surface, and made a hydrogel layer on the surface. The PEGDMA macromonomers within the physically crosslinked hydrogel were covalently crosslinked in the next step to form a stiff interpenetrating network. The hydrogel network bound to the scaffold surface through reaction of double bonds of PEGDMA macromonomers and double bonds of APMA grafted to the scaffold surface. The covalent binding between reactive functional groups of PEGDMA and those of scaffold surface during the crosslinking reaction, increased the hydrogel adhesion to the surface of PCL-TCP scaffolds. Adhesion of polymer networks to rigid surfaces increases when functional groups on the surface link to the polymer network. For instance, the adhesion between a PEGDA/alginate IPN hydrogel and glass, ceramics, titanium or aluminum significantly increased with modifying the surface with reactive 3-(trimethoxysilyl) propyl methacrylate (TMSPMA) and covalent anchoring the hydrogel to the surface.

The hydrogel layer thickness was directly correlated with the total amount of released calcium ion from the surface and could be tailored with altering processing parameters including NaOH treatment time and CaCl₂) concentration in the treatment solution. An increase in the total amount of released calcium ion from the scaffolds with raising NaOH treatment time was due to an improved surface hydrophilicity and roughness, hence higher absorption of CaCl₂ solution on the PCL-TCP surface. Likewise, an increase in the total amount of released calcium ions from the scaffolds with raising CaCl₂) concentration in the treatment solution was due to a larger calcium deposition on the surface.

Despite an elevated hydrogel loading, the hydrogel thickness did not dramatically change with increasing the scaffold porosity. Therefore, the pore size of porous HyTECs could be tuned by changing the scaffold porosity. The size of interconnected pores in bone tissue engineering scaffolds should be at least 100 μm for cell infiltration, bone ingrowth, and vascularization. However, cell migration and bone ingrowth is optimal when the pore size was larger than 100 μm. For example, the adhesion and proliferation of osteoblasts on collagen-glycosaminoglycan (CG) scaffolds with pore size greater than 300 μm were higher than those scaffolds with pore size smaller than 200 μm [24]. When porous poly(ether ester) block-copolymer scaffolds were implanted into the dorsal skinfold chamber of balb/c mice, vessel ingrowth was faster for the scaffolds with large pores (250-300 μm) compared to those with medium pores (75-212 μm) or small pores (20-75 μm). In this invention, the scaffolds pore size after hydrogel loading ranged from 140 μm to 300 μm, 480 μm, 1100 μm when the porosity of pristine scaffolds increased from 50% to 60%, 70% and 80%. Although the pore size of all hydrogel loaded scaffolds was larger than 100 μm, those scaffolds with minimum 60% porosity and 300 μm or larger pore size after hydrogel loading would be optimal for future in-vivo experiments, based on the aforementioned published reports.

Results presented herein showed that BMP2 was released from alginate/PEG-based hydrogel loaded porous PCL-TCP scaffolds over 35 days. It has been demonstrated that a BMP2 loaded alginate/PEG based hydrogel with a sustained release of BMP2 in-vitro stimulated ectopic bone nodule formation in mice. Therefore, incorporation of BMP2-laden alginate/PEG-based hydrogel imparts osteo-inductivity to osteoconductive 3D printed porous PCL-TCP scaffolds and potentially enhances and accelerates bone formation. A lower amount of released protein at higher PEGDMA concentrations (FIG. 16G) was caused by a growth in the crosslink density and a drop in the mesh size of the polymer network.

Freeze-drying facilitates storage/transportation and increases the shelf-life of bioactive products. Results presented herein showed that freeze-drying did not adversely influence the mechanical properties of the scaffolds and characteristics of the hydrogel layer but reduced the release of bioactive BMP2. PCL-TCP scaffolds are heat sensitive and E-beam irradiation is considered a reliable method for terminal sterilization of heat sensitive materials. It was also shown that E-beam sterilization did not adversely affect mechanical properties and degradation kinetics of PCL-TCP scaffolds. The results of the present invention revealed that E-beam sterilization reduced the release of bioactive BMP2 from freeze-dried BMP2-laden porous HyTECs.

The results presented herein further showed that osteogenic differentiation of hMSCs and C3H10 cells was higher when the cells were exposed to BMP2 released from porous HyTECs compared to 3-day exposure of cells to BMP2 dissolved in the medium at the same dose as BMP2 loading in hydrogel. The higher osteogenic differentiation of cells exposed to BMP2 releasing scaffolds compared to 3-day exposure of cells to BMP2 was due to the time-dependent osteo-inductivity of BMP2. Osteo-inductivity of BMP2 protein is dose- and time-dependent. For instance, the ALP activity of hMSCs exposed to slow BMP2 releasing electrospun PCL/PEG mats was significantly higher than that of hMSCs exposed to fast BMP2 releasing mats.

The method of this invention could be used for loading a wide range of hydrogels on porous or non-porous polyester-based constructs. Therefore, in addition to the porous scaffolds, the inventors investigated the efficacy of the method for making non-porous HyTECs with a bioresorbable hydrogel for sustained release of BMP2 protein. To improve the integration of hydrogel with the non-porous rod, freezing/thawing was used after NaOH treatment to increase the surface roughness. The results showed that freezing/thawing increased calcium deposition on the surface of the rods (not shown). GelMA and PEGDMA were used as macromonomer and crosslinker, respectively. HepMA was used for prolonging the release of BMP2, due to a high affinity of heparin to BMP2. It has been shown that addition of HepMA to an alginate-based hydrogel extended the release kinetics of BMP2 and improved subcutaneous bone formation in mice. In addition, photo-initiation was used for covalent crosslinking of the hydrogel on non-porous rods instead of chemical-initiation that was used for crosslinking of hydrogels in porous scaffolds. Chemically initiated crosslinking was used in porous scaffolds because the penetration of light into the central parts of the scaffold might be limited. The release of BMP2 (as a model protein) from non-porous HyTECs showed that the method can be used for sustained delivery of proteins along with non-porous implants. Total amount of released enzymatically active BMP2 from hydrogel loaded rods after freeze-drying and single E-beam sterilization was only 28%. That might be attributed to denaturation of BMP2 or crosslinking of a GelMA based network with BMP2 under highly intense radiation. However, the amount of released active BMP2 almost doubled with splitting a highly intense E-beam dose (25 kGy) to two doses (12.5 kGy) with lower intensity. The inventors also showed that the method described herein could be used for loading live cell-laden hydrogels on scaffolds. The biodegradable hydrogel layer was thick enough to accommodate cells and the cells were viable and proliferating. The described method could be particularly useful for cell loading on implants.

### BENEFIT

The present invention claims the benefit, or priority, to U.S. Provisional Applications 63/289,431 filed Dec. 14, 2021, 63/304,216 filed Jan. 28, 2022, 63/289,447 filed Dec. 14, 2021, and 63/304,207 filed Jan. 28, 2022 all of which are incorporated herein by reference for all that they teach.

What is claimed is:

1. A method of forming a tissue engineering construct, comprising:
(a) having a scaffold with a surface and a surface area;
(b) treating the surface of the scaffold to increase the surface area of the scaffold;
(c) optionally preparing the surface area of the scaffold to facilitate a chemical cross-linking to the surface area by coating the surface area with covalently linkable molecules;
(d) preparing the surface area of the scaffold to facilitate surface-initiated physical cross-linking by depositing a salt onto the surface area or the optionally coated surface area;
(e) preparing a hydrogel precursor solution containing charged polymers, optionally covalently reactive macromonomers, an initiator and biologics;
(f) forming a physically cross-linked hydrophilic hydrogel network onto the surface of the scaffold by immersing the prepared scaffold into a hydrogel precursor solution, wherein the forming is controlled by a release of salt-ions from the surface area and physically cross-linking the charged polymers with the released salt-ions, and wherein during the formation the biologics becomes trapped and thereby hosted within the physically cross-linked hydrogel network;
(g) removing the scaffold with the physically cross-linked hydrophilic hydrogel network from the hydrogel precursor solution;
(h) chemically cross-linking the covalently reactive macromonomers within the physically cross-linked hydrophilic hydrogel network to strengthen the physically cross-linked hydrophilic hydrogel network itself and to the scaffold; and
(i) optionally chemical cross-linking the coated covalently linkable molecules with the covalently reactive macromonomers to increase adhesion of the chemically and physically cross-linked hydrophilic hydrogel network to the scaffold.

2. The method as set forth in claim 1, further comprising freezing or freeze-drying of the tissue engineering construct.

3. The method as set forth in claim 1, further comprising further treating the surface area of the scaffold to increase the hydrophilicity, roughness, or a combination thereof of the surface of the scaffold.

4. The method as set forth in claim 1, wherein the scaffold has an interconnected porous structure, and wherein the methods steps are controlled for the hydrophilic hydrogel network to be physically and chemically crosslinked and chemically bound to the interconnected porous structure of the scaffold, and wherein the pores of the interconnected porous structure can be preserved by the method steps to allow the pores to also be house the biologics.

5. The method as set forth in claim 1, further comprising coating the tissue engineering construct with one or more coating layers.

6. The method as set forth in claim 5, wherein the scaffold has an interconnected porous structure and wherein the coating controls pore size of the interconnected porous structure.

7. A tissue engineering construct, comprising:
(a) a scaffold with a surface and a treated surface area for increased surface area;
(b) a hydrophilic hydrogel network physically cross-linked via charged polymers and salt-ions onto the treated surface area;
(c) biologics trapped and thereby hosted within the physically cross-linked hydrogel network; and
(d) covalently reactive macromonomers chemically cross-linked within the physically cross-linked hydrophilic hydrogel network to strengthen the physically cross-linked hydrophilic hydrogel network itself and to the scaffold.

8. The tissue engineering construct as set forth in claim 7, wherein the surface area is coated with covalently linkable molecules which are chemical cross-linked with the covalently reactive macromonomers to increase adhesion of the chemically and physically cross-linked hydrophilic hydrogel network to the scaffold.

9. The tissue engineering construct as set forth in claim 7, wherein the tissue engineering construct has one or more coating layers.

10. The tissue engineering construct as set forth in claim 7, wherein the scaffold is an interconnected porous scaffold and wherein the biologics is hosted with pores of the interconnected porous scaffold.
